(19) United States
(12) Patent Application Publication
(10) Pub. No.: US 2025/0252467 A1
(43) Pub. Date: Aug. 7, 2025

(54) DRUG-COST DECISION SUPPORT METHODS AND APPARATUSES FOR PATIENT EMPOWERMENT

(71) Applicant: Gemini Health LLC, Sausalito, CA (US)

(72) Inventors: Edward FOTSCH, Sausalito, CA (US); Christopher LeCOMTE, Rootstown, OH (US); Roger PINSONNEAULT, Johns Creek, GA (US)

(21) Appl. No.: 19/047,620

(22) Filed: Feb. 6, 2025

Related U.S. Application Data
(60) Provisional application No. 63/550,111, filed on Feb. 6, 2024.

(51) Int. Cl. G06Q 30/0283; G16H 10/60; G16H 20/10

(52) U.S. Cl. CPC G06Q 30/0283; G16H 10/60; G16H 20/10

(57) ABSTRACT

Described herein are systems and methods for implementing a drug-cost decision-support having: an enrollment engine configured to enroll a patient and register patient-related information; a drug and savings report engine configured to generate one or more of: a drug savings report (DSR) and drug determination policies (DDP) providing cost and alternatives for one or more drugs prescribed for the patient; an outreach engine configured to respond to a medication trigger with the generated one or more of the DSR and DDP; and a graphical user interface (GUI) portal configured to display the DSR and DDP at the GUI and provide an ordering interface configured to order the one or more drugs. Thus, the platform enables patients to choose their preferred pharmacy for prescription routing, ensuring cost savings are delivered upfront and providing transparent out-of-pocket cost information before a pharmacy processes the prescription. Other examples are described.

### CLAIM OF PRIORITY

This application claims the benefit of U.S. Provisional Patent Application No. 63/550,111 entitled “MY RX GUIDE,” filed on Feb. 6, 2024, the entire contents of which are incorporated herein by reference.

### BACKGROUND

Drug prescription drug costs in the United States continue to rise driven by multiple factors including a lack of cost transparency for prescribers and patients. Cost transparency is foundational for competition and resultant lower costs. In recent years, prescribers have begun seeing patient-specific drug costs at the point of prescribing in their electronic health records (EHR), but problematically this information is inconsistent and typically lacks lower cost alternative drugs and pharmacies depending upon the payer providing insurance to the specific patient. Even when accurate drug and alternative costs are provided to prescribers, workflow challenges, lack of financial motivation and lack of patient awareness of the cost of the drugs at time of prescribing, or when engaging healthcare providers, limit the selection of the most cost-effective drug and pharmacy. Prescribers are often the sole decision-maker for selecting medications to prescribe and payers, employers, and patients paying for medications often lack awareness and thus a role in the decision making about lower cost drug options.

What is needed is a platform to directly and automatically engage a patient in a convenient and digital manner to offer cost-effective drug pricing, alternative drug pricing, insurance coverage information, and alternative pharmacy information. Additionally, there is a need for a solution that empower patients to seamlessly transfer unfilled prescriptions to their preferred pharmacy, request lower-cost alternatives, or place prescriptions on hold, reducing administrative burdens and ensuring greater accessibility to affordable medications.

### SUMMARY OF THE DISCLOSURE

Described herein are systems and methods for implementing a drug-cost decision support platform having: an enrollment engine configured to enroll a patient and register patient-related information including one or more of: patient contact information, insurance and eligibility information, clinic information, hospital information, healthcare provider information (which may include the patient's primary care provider), medical appointment information, hospital stay information, medication information including electronic health record (EHR) information, prescription information and health information for the patient; a drug and savings report engine configured to generate one or more of: a drug savings report (DSR) and drug determination policies (DDP) providing cost and alternatives for one or more drugs prescribed for the patient; an outreach engine configured to respond to a medication trigger with the generated one or more of the DSR and DDP; and a graphical user interface (GUI) portal configured to display the one or more of the DSR and DDP and provide an ordering interface configured to order the one or more drugs.

According to certain examples, the GUI portal configured to display the one or more of the DSR and DDP includes one or more of: a member portal and an interface in the EHR; further in which the GUI portal is accessed via a link sent via one or more of: a text message, a phone call, e-mail and mobile app. One or more of patient spoken language, patient printed language and engagement acknowledgement may be used to display the one or more of the DSR and DDP.

According to certain examples, the platform further includes a matching engine configured to evaluate whether the patient-related information including the patient contact information and healthcare provider information matches pharmacy claim information of a health plan for the patient.

According to certain examples, the platform includes a rules engine configured to incorporate clinical and financial rules in generating the one or more of the DSR and DDP.

According to certain examples, the medication trigger is based on one or more of: the patient-related information, a prescription order, a real-time prescription benefit request, an EHR update, a request on behalf of the patient, a request on behalf of the healthcare provider, and a message from the platform to a pharmacist.

According to certain examples, the one or more of the DSR and DDP further includes information from retailers, mail order, specialty pharmacies, and third-party pharmacies; in which the one or more of the DSR and DDP further includes payment and financial assistance information.

According to certain examples, the platform further includes a reporting engine configured to report one or more of: patient satisfaction, patient utilization of the platform and impact of the platform in real-time prescribing behavior of healthcare providers based on reduced patient call-back for prescribing the one or more medications.

According to certain examples, the platform further includes a transaction mapping engine configured to match the one or more of the DSR and DDP results to prescription claims to gauge impact of the platform.

According to certain examples, the platform further includes a billing services integration engine configured to integrate with and send notification to billing and prior authorization services for medications billable to one or more of a pharmacy and medical benefit coverage eligibility.

According to certain examples, the drug and savings report engines may be further configured to assess and report status of one or more of: patient coverage, patient deductible, prior authorization, alternative drugs not requiring prior authorization and prescription ready for pick-up status.

According to certain examples, the prescription information includes a preferred pharmacy for the patient the platform, further in which the platform includes a mechanism for one or more of: transferring unfilled prescriptions to a preferred pharmacy, requesting lower-cost drug alternatives, and placing prescriptions on hold. The platform may also further include an interface configured for a pharmacist to view, monitor and interact with patients enrolled in the platform.

In yet other aspects, there is a method for expedited and cost-effective digital prescription and ordering of medications, the method including: registering a patient and related information of the patient via an enrollment engine of a drug-cost decision-support platform; receiving a medication trigger for the patient at the platform; determining patient eligibility for one or more drugs; assessing and generating, via a drug cost and savings report engine, one or more of a drug savings report (DSR) and drug determination policies (DDP) providing cost and alternative drug data for the one or more drugs; sending the DSR to a graphical user interface (GUI) portal configured to receive input for ordering the one or more drugs; sending, via an outreach engine, a request for user input at the GUI portal; receiving the user input at the GUI portal; and ordering the one or more drugs based on the input received at the GUI portal.

In certain examples of this method, receiving the medication trigger is based on one or more of: the related information of the patient, a prescription order, a real-time prescription benefit request, an EHR update, a request on behalf of the patient, a request on behalf of the healthcare provider, and a message from the platform to a pharmacist.

In certain examples of this method, the method further includes receiving the user input from one or more of: the patient, a healthcare provider, and a pharmacist.

In certain examples of this method, the method further includes accessing the GUI portal to provide the user input via one or more of: a member portal configured for the patient to access the one or more of the DSR and DDP and an interface in the EHR configured for a healthcare provider to access the one or more of the DSR and DDP; further in which accessing the GUI portal is achieved via a link sent via one or more of: a text message, a phone call and e-mail.

In certain examples of this method, the method further includes matching, via a matching engine, the related information of the patient including patient contact information and healthcare provider information with pharmacy claim information of a health plan for the patient.

In certain examples of this method, the method further includes incorporating clinical and financial rules in assessing and generating the DDP or DSR via a rules engine.

In certain examples of this method, the method further includes matching the one or more of the DSR and DDP to prescription claims via a transaction mapping engine to gauge impact of the platform.

In certain examples of this method, the method further includes integrating and notifying billing and prior authorization services via a billing services integration engine.

In certain examples of this method, the method further includes assessing and reporting status of one or more of: patient coverage, patient deductible, prior authorization, alternative drugs not requiring prior authorization and prescription ready for pick-up status via the drug cost and savings report engine.

In still other aspects, there is another method executing within a system of a host organization, the system having a processor and a memory therein to execute instructions within the system, in which the method includes: registering a patient and related information of the patient via an enrollment engine of a drug-cost decision-support platform; receiving a medication trigger for the patient at the platform; determining patient eligibility for one or more drugs; assessing and generating, via a drug cost and savings report engine (drug and savings report engine), a drug savings report (DSR) and drug determination policies (DDP) providing cost and alternative drug data for the one or more drugs; sending the one or more of the DSR and DDP to a graphical user interface (GUI) portal configured to receive input for ordering the one or more drugs; sending, via an outreach engine, a request for user input at the GUI portal; receiving the user input at the GUI portal; and ordering the one or more drugs based on the input received at the GUI portal.

Further still in other examples, there is a non-transitory computer readable storage media having instructions stored thereon that, when executed by a processor of a system, the instructions cause the system to perform operations including: registering a patient and related information of the patient via an enrollment engine of a drug-cost decision-support platform; receiving a medication trigger for the patient at the platform; determining patient eligibility for one or more drugs; assessing and generating, via a drug cost and savings report engine, one or more of a drug savings report (DSR) and drug determination policies (DDP) providing cost and alternative drug data for the one or more drugs; sending the one or more of the DSR and DDP to a graphical user interface (GUI) portal configured to receive input for ordering the one or more drugs; sending, via an outreach engine, a request for user input at the GUI portal; receiving the user input at the GUI portal; and ordering the one or more drugs based on the input received at the GUI portal.

All of the methods and apparatuses described herein, in any combination, are herein contemplated and can be used to achieve the benefits as described herein.

### BRIEF DESCRIPTION OF THE DRAWINGS

A better understanding of the features and advantages of the methods and apparatuses described herein will be obtained by reference to the following detailed description that sets forth illustrative embodiments, and the accompanying drawings of which:

FIG. 1 depicts an exemplary architecture for a drug-cost decision-support platform in accordance with described examples.

FIG. 2 depicts a tablet computing device and a hand-held smartphone each having a circuitry integrated therein for interfacing with a GUI portal of the drug-cost decision-support platform as described in accordance with the examples.

FIG. 3 depicts a general workflow for a prescription notification using the drug-cost decision-support platform.

FIG. 4A depicts various views of a GUI of the drug-cost decision-support platform configured for patient-member usage.

FIG. 4B depicts prescription routing and other functionality of the drug-cost decision-support platform.

FIG. 5A depicts an exemplary workflow for the drug-cost decision-support platform at prescribing.

FIG. 5B depicts another workflow of the drug-cost decision support platform at prescribing including user interactions.

FIG. 5C depicts a workflow of the drug-cost decision support platform at an electronic health record (EHR).

FIG. 5D depicts another workflow of the drug-cost decision support platform at an electronic health record (EHR) including user interactions.

FIG. 6 illustrates a diagrammatic representation of a machine in the exemplary form of a computer system configured to operate the drug-cost decision support platform, in accordance with one example.

FIG. 7 is a flow chart illustrating a method for expedited and cost-effective prescription and ordering of medications using the drug-cost decision support platform.

FIGS. 8A-8B illustrate a workflow for MRG Patient-Member Engagement.

FIG. 8C illustrates MRG-aRTPB notifications under an existing process.

FIGS. 8D-8E illustrates MRG-aRTPB notifications under the Platform.

FIG. 8F illustrates a workflow for Platform Satisfaction.

### DETAILED DESCRIPTION

Described herein are systems and method for a drug-cost decision support platform (“Platform”), in some examples known as medical Rx guide platform (e.g., “MRG”), that may provide drug cost information including lower cost alternative drug and pharmacy information to health plan members to help reduce prescription drug costs. In certain examples, the Platform messages to health plan members may be triggered by healthcare workflows, such as a new prescription created via electronic prescribing in electronic health records. This allows patients for the first time to know the cost of a new prescription before they go to the pharmacy and to know if a lower cost alterative drug or pharmacy is available before their provider sends the prescription to a pharmacy.

Alternatively, the Platform message may be triggered by a new request for Prior Authorization for a medication. This is important because over half of prescriptions for specialty medications are NOT ordered using electronic prescribing in electronic health records; a fact which is not known or understood by most health plan and related industry experts. Specialty medications coverage by the Platform and the related Prior Authorization integration is critical because these drugs in aggregate make up roughly half of all prescription spending in the United States. Alternatively, a Platform message may be triggered by upcoming patient appointments with a provider using an integration with eligibility software and EDI network functionality known 270/271 queries from electronic health records to health plans. Alternatively, a Platform message may be triggered on-demand by patients.

The Platform may thus directly and interactively digitally engage patients with drug and alternative cost information and coverage information to help reduce prescription drug costs. For example, the Platform may be activated by a provider prescribing in Electronic Health Records (EHRs) to initiate patient outreach delivering drug and alternative cost information in real-time, customized for the individual patient, potentially while the patient is still in the exam room and well-prior to the patient filling the prescription. The patient (which may become a patient-member once the patient is enrolled with the Platform), can then engage or re-engage the prescriber to select the most cost-effective medications and pharmacies. The patient-member can also request assistance in engaging the prescriber from a pharmacist or similar support person who can then engage the prescriber on the patient's behalf to discuss lower cost alternative medications. The patient-member can also respond to the outreach to query for additional information including more alternative pharmacies or to ask for a list of all of their existing medications including lower cost alternative drugs and pharmacies so that the patient can discuss all of their medications with their provider(s). Additionally, the patient-member may choose to utilize RxRoute, a service within the MRG suite for transferring unfilled prescriptions to their preferred pharmacy, request lower-cost alternatives, or place prescriptions on hold, without an intervening prescriber. The transfer function is important and unique because standard ePrescribing network flows do not allow patients to move their new prescriptions without re-engaging their prescribers, which is challenging for both the patient and the prescriber. Related patient-member contact information can also be used to promote other drug related outreach through the Platform including Drugs and Savings Reports (DSR) and the Platform's MedOps interactive pharmacists' desktop.

Described herein are systems and methods for implementing a uniform application user interface for the Platform across a hosted computing environment, such as an on-demand or cloud computing environment which utilizes multi-tenant database technologies, client-server technologies, traditional database technologies, or other computing architecture in support of the hosted computing environment. Such an exemplary system may include, for example: a processor and a memory to execute instructions at the system; a foundation layer to define a plurality of components; the plurality of components, each to define one or more features to be consumed by an arbitrary application built from the features; wherein the one or more features are to each incorporate one or more of the components defined by the foundation layer and further wherein each of the one or more features have visibility to one or more interfaces available for the respective features to connect with but have no visibility to or about any arbitrary application that will consume them; a glue logic layer to link the features to the arbitrary application built from the features, wherein the arbitrary application built from the features has a one-way view of the features consumed through the glue logic layer without permitting the features visibility to or about the arbitrary application built; and wherein the arbitrary application built from the features is to execute within the host organization.

The systems and methods described herein enable a predictable and cohesive relationship between a UI architecture and underlying features and functionality which operates within a large scale and highly used hosted computing environment supporting multiple distinct tenants.

Certain embodiments operate within a hosted computing environment, also referred to as a provider of on-demand services, on-demand database services, cloud computing services, or simply a host organization that provides services to subscribing customer organizations. Such host organizations utilize various technologies to service many different tenants (e.g., customer organizations and their users) simultaneously. Such technologies may include, for example, client-server implementations, computing grids, computing pods or pools of work machines, traditional databases, single tenancy database systems and/or multi-tenant database systems. A multi-tenant database system in particular operates to store data on behalf of a multitude of subscribers, each being a “tenant” of the database system, hence the term multi-tenant database system. Many subscribers (e.g., users or tenants) utilize the computing technologies of the host organization to access analytics, charts, views, reports, and other such data which is stored within the servers, systems, databases, and multi-tenant database system of the host organization. For instance, a sales team may utilize sales data stored within such a system.

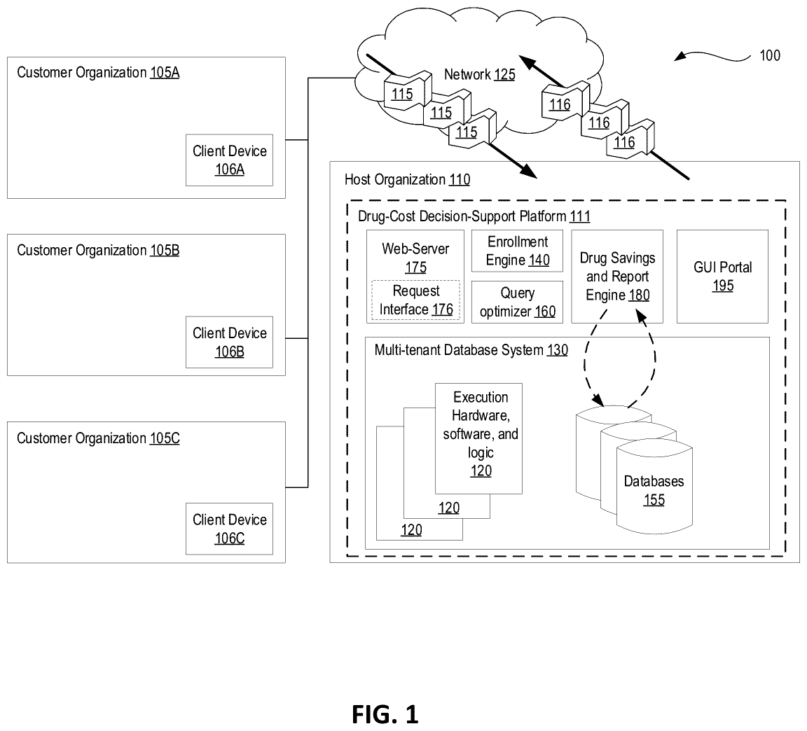

FIG. 1 depicts an exemplary architecture 100 in accordance with described embodiments. In one embodiment, the drug-cost decision support platform (Platform) 111 is communicably interfaced with a plurality of client devices 106A-C (e.g., such as mobile devices, smart phones, tablets, PCs, etc. used by patients, providers, pharmacists, etc.) through host organization 110. In one embodiment, a multi-tenant database system 130 includes databases 155, for example, to store tables, datasets, and underlying database records with user data on behalf of customer organizations 105A-C (e.g., tenants of the multi-tenant database system 130 or their affiliated users).

Multi-tenant database system 130 includes a plurality of underlying hardware, software, and logic elements 120 that implement database functionality and a code execution environment within the host organization 110. In accordance with one embodiment, multi-tenant database system 130 further implements databases 155 to service database queries and other data interactions with the databases 155. The hardware, software, and logic elements 120 of the multi-tenant database system 130 are separate and distinct from a plurality of customer organizations (105A, 105B, and 105C) which utilize the services provided by the host organization 110 by communicably interfacing to the host organization 110 via network 125. In such a way, host organization 110 may implement on-demand services, on-demand database services or cloud computing services to subscribing customer organizations 105A-C.

Host organization 110 receives input and other requests 115 from a plurality of customer organizations 105A-C via network 125 (such as a public Internet). For example, incoming database queries, API requests, interactions with displayed graphical user interfaces and displays at the client devices 106A-C, or other inputs may be received from the customer organizations 105A-C to be processed against the multi-tenant database system 130. In certain embodiments, the inputs and requests 115 from the customer organizations 105A-C may include custom code, features, and functionality to be hosted and executed within the host organization 110 on behalf of such customer organizations 105A-C. In such embodiments, responses 116 may constitute data records, reports, analytics, charts, or other information provided by either the customer organizations' 105A-C previously provided customized code, features, and functionality or may be provided by code, features, and functionality made accessible to the customer organizations 105A-C as a service, or may be some combination of both.

In one embodiment, each customer organization 105A-C is an entity selected from the group consisting of: a separate and distinct remote organization, an organizational group within the host organization 110, a business partner of the host organization 110, or a customer organization 105A-C that subscribes to cloud computing services provided by the host organization 110.

In one embodiment, requests 115 are received at, or submitted to, a web-server 175 within host organization 110. Host organization 110 may receive a variety of requests for processing by the host organization 110 and its multi-tenant database system 130. Incoming requests 115 received at web-server 175 may specify which services from the host organization 110 are to be provided, such as query requests, search request, status requests, database transactions, graphical user interface requests and interactions, processing requests to retrieve, update, or store data on behalf of one of the customer organizations 105A-C, code execution requests, and so forth. Web-server 175 may be responsible for receiving requests 115 from various customer organizations 105A-C via network 125 and provide a web-based interface or other graphical displays to an end-user client device 106A-C or machine originating such data requests 115.

Host organization 110 may implement a request interface 176 via web-server 175 or as a stand-alone interface to receive requests packets or other requests 115 from the client devices 106A-C. Request interface 176 further supports the return of response packets or other replies and responses 116 in an outgoing direction from host organization 110 to the client devices 106A-C.

The present disclosure may allow for improved operating speeds of the components of Platform 111 including engines and interfaces described below.

Enrollment engine 140 operates on behalf of the host organization to provide outreach and enrollment for patients (users) to access Platform 111. Enrollment engine 140 may be configured to enroll and register patient-related information including one or more of: patient contact information, insurance eligibility information, clinical information, hospital information, healthcare provider information, medical appointment information, hospital stay information, medication information including electronic health record (EHR) information, prescription information and health information for a patient.

In certain examples, there may be a separated or integrated outreach engine configured to respond to a medication trigger with the drug savings report (DSR) and/or drug determination policies (DDP) generated by Drug Savings and Report Engine 180 described below.

The medical trigger may be based on one or more of: the patient-related information, a prescription order, a real-time prescription benefit request, an EHR update, a request on behalf of the patient, a request on behalf of the healthcare provider, and a message from the platform to a pharmacist.

In certain examples, the DSR and/or DDP may further include information from retailers, mail order, specialty pharmacies, and third-party pharmacies, as well as payment and financial assistance information.

Aspects of Platform 111 member/patient outreach executed by enrollment engine 140 may include:
1. Patient-members being offered Platform enrollment at the time of enrolling a health plan.
2. Patient-members whose email or cell phone numbers for texting are known to a payer being offered enrollment to the Platform digitally.
3. Patient-members whose email or cell numbers are not known to a payer being offered enrollment via mail and marketing/press outreach including a Know-Before-You-Go campaign to drive prescription drug cost awareness.
4. Employers having contact information for their covered employees.
5. Payers electing to include financial incentives for senior enrollment as is permitted via CMS-4190.
6. Patient-members being able to opt out of Platform membership at any time.

Aspects of Platform provider outreach may include providers and provider groups being advised in-advance that their enrollment with the Platform will begin on a certain date and that their patients will know the cost of medications quickly after prescriptions are created via email and text. Providers may wish to increase focus on Real-Time Prescription Benefit (RTPB) technology at prescribing in their EHRs to avoid patient call-backs.

Aspects of employer outreach may include covered employers being notified of the upcoming availability of the Platform and offering enrollment of their employees subject to opt-out.

Drug Savings and Report Engine 180 provides functionality to pass queries from web-server 175 into the multi-tenant database system 130 for execution against the databases 155 or other data stores of the host organization's Platform 111 to generate drugs savings report(s) (DSR) and drug determination policies (DDP) to provide cost and alternative for one or more drugs prescribed for a patient.

Drug Savings and Report Engine 180 may be configured to assess and report status of one or more of: patient coverage, patient deductible, prior authorization, alternative drugs not requiring prior authorization and prescription ready for pick-up status.

In one embodiment, the Drug Savings and Report Engine 180 implements an Application Programming Interface (API) through which queries may be executed against the databases 155 or other data stores including queries arriving from a foundational layer of the GUI Portal or Drug Savings Report at an Electronic health Record (DSR-E) 195. Query optimizer 160 performs query translation and optimization, for instance, on behalf of other functionality such as functionality of a graphical interface which possesses sufficient information to architect a query (e.g., identifying parameters, targets, tables, records, rows, actions, etc.) yet lacks the necessary logic to actually construct the appropriate query syntax into the databases 155 of the multi-tenant database system 130. In other instances, query optimizer 160 modifies a submitted query to optimize its execution within the host organization without affecting the resulting dataset returned responsive to such an optimized query. Various databases that the Platform may link to may include, for example, Ambulatory Medical Records (AMR) of a patient-member's outpatient medical records or specialized SPS databases withing a larger EHR system such as a database for Surgical Procedure Services or Specialty Patient Services.

In certain examples, there may be a matching engine configured to evaluate whether the patient-related information including the patient contact information and healthcare provider information matches pharmacy claim information of a health plan for the patient.

In certain examples, there may be a rules engine configured to incorporate clinical and financial rules in generating the one or more of the DSR and DDP.

In certain examples, a reporting engine may be configured to report one or more of patient satisfaction and impact of the platform in real-time prescribing behavior of healthcare providers based on reduced patient call-back for prescribing the one or more medications.

In certain examples, there may be a transaction mapping engine configured to match the one or more of the DSR and DDP results to prescription claims to gauge impact of the platform.

In certain examples, there may be a billing services integration engine configured to integrate with and send notification to billing and Prior Authorization services for medications billable to one or more of a pharmacy and medical benefit coverage eligibility.

UI Framework 195 enables a predictable and cohesive relationship between a UI architecture and underlying features and functionality, whether provided by the host organization 110 or the customer organizations 105A-C, permitting the UI architecture and features and functionality to operate within the large scale and highly used multi-tenant environment established by the computing architecture of the host organization 110. Use and implementation of such a UI framework 195, as will be described in additional detail below, further avoids the stickiness and coupling between the features and functionality and the UI architecture which is problematic with conventionally available solutions.

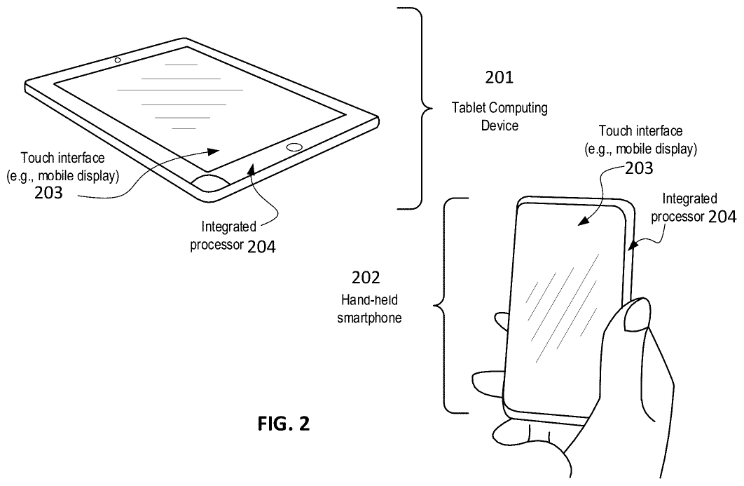

FIG. 2 depicts a tablet computing device 201 and a hand-held smartphone 202 each having a circuitry integrated therein as described in accordance with the embodiments. As depicted, each of the tablet computing device 201 and the hand-held smartphone 202 include a touch graphical user interface (GUI) 203 (e.g., a touchscreen or touch sensitive display) and an integrated processor 204 in accordance with disclosed embodiments.

For example, in one embodiment, a system embodies a tablet computing device 201 or a hand-held smartphone 202, in which a display unit of the system includes a touch GUI 203 for the tablet or the smartphone and further in which memory and an integrated circuit operating as an integrated processor are incorporated into the tablet or smartphone, in which the integrated processor implements one or more of the embodiments described herein. In one embodiment, the integrated circuit described above or the depicted integrated processor of the tablet or smartphone is an integrated silicon processor functioning as a central processing unit (CPU) and/or a Graphics Processing Unit (GPU) for a tablet computing device or a smartphone.

In certain examples, touch GUI 203 may be configured to display, via a GUI portal, the one or more of the DSR and DDP and further provide an ordering interface configured to order one or more drugs prescribed for a patient.

In yet other examples, the GUI portal may include one or more of a member portal and an interface in the EHR. The GUI portal may be accessed via a link sent by text message, a phone call, e-mail or mobile app. Patient spoken language, patient printed language, and engagement acknowledgment may be used to display the DSR and/or DDP.

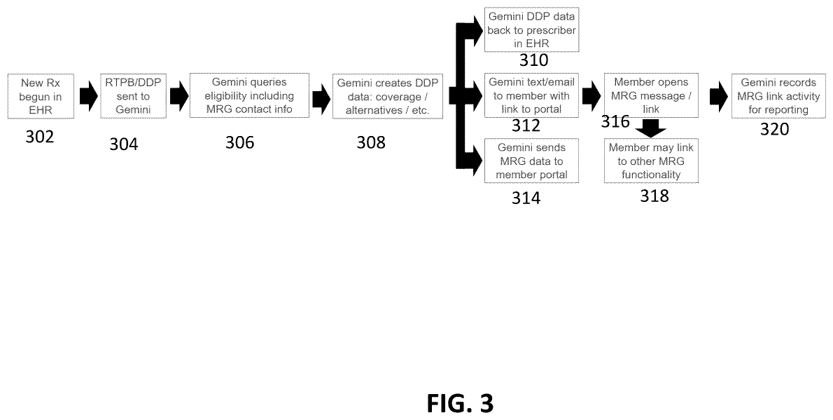

FIG. 3 depicts a general workflow for a prescription notification using the drug-cost decision-support platform.

As shown here, a new prescription may begin in an EHR 302, followed by real-time prescription benefit (RTPB) and drug determination policies (DDP) sent to the Platform 304. The Platform may then query eligibility including contact information 306. The platform may then created drug determination policy data including coverage, alternatives, etc. 308. In certain examples, following this step, possible subsequent steps may include one or more of: DDP data being sent back to a prescriber via the EHR 210, the Platform texting or e-mailing a patient or patient-member with a link to a portal to access the Platform 312, and the Platform sending data to a member portal 314.

Pursuant to the Platform texting or e-mailing the patient or patient-member with a link to the portal to access the Platform 312, the member may open the Platform message/link 316, which in certain examples may result in either: the Platform recording activity related to the link for reporting 320 or the member may link to other Platform functionality 318.

### Platform Workflows

In certain examples, messages triggered by ePrescribing may include:

### 1. Platform Trigger Messages

Platform trigger messages may be activated when:
a. A patient is enrolled in MRG services.
b. The Patient's Prescriber initiates New Rx (NRx) in EHR triggering RTPB transaction to be sent to the Platform.

Alternatively, Platform messages could be triggered by a Prior Authorization (PA) request including ePA if the medication does not trigger an RTPB such as an infused medication or medication covered under the medical benefit.

Alternatively, an e-Prescribing NewRx transaction initiated by a provider in the EHR can trigger a Platform message or transaction.

Alternatively, a new Prior Authorization for a medication initiated by a provider can trigger a Platform message or transaction.

Alternatively, member appointment or engagement with a provider can trigger an Platform transaction or message that includes all existing medications.

Alternatively, analysis including AI of the pharmacy claims for a given patient can trigger a Platform transaction or message that includes all existing medications.

### 2. Platform Matching

The Platform may match members in RTPB transactions to patient-members enrolled in the Platform.

### 3. Platform Rules Engine

The Platform may use a customizable rules engine to determine if Platform access is sent and/or if Platform data will be modified for a specific patient-member.

The Platform may allow patient-members to suppress one or more specific alternatives in their DSRs for a specified period of time.

### 4. RTPB/Platform Data

The Platform may generate NRx information (NRx-i) including the cost of the drug selected at the chosen pharmacy, any prior authorization or step therapy or coverage information, and lower cost alternative drugs or fulfillment including (i.) 90 Days' Supply at Retail, (ii.) Mail Order, (iii.) Specialty Pharmacy, and/or 3^{rd} parties such as CivicaScript, CostPlus/Cuban drugs etc. Information can include clinical information for members as well.

Platform data can also include Cash Card options, payer-approved patient financial assistance, Shared Patient Savings and other member benefits.

### 5. Platform Member Outreach

The Platform may send a text or email to the patient-member enrolled in a Platform program with a hypertext link to the NRx-i. Patient-members who click on the link will be presented with NRx-i as well as the option to view all their current medications along with lower cost alternative drugs and pharmacies on a web page which can be hosted by the Platform, host organization, or payer using payer logos and UI/UX.

Alternatively, Platform member outreach can be delivered via telephone messages directing members to contact member services, get data via telephone or login to Platform/member portal or download the related Platform or payer Mobile App.

### 6. Platform Transaction Mapping

Platform transactions which result in NRx-i viewed by members may be recorded by the Platform or host organization for reporting purposes and matched to downstream claims to gauge impact.

### 7. Platform Reporting

The Platform may be configured to report monthly on the impact of the Platform as measured in changes to prescriptions which reduce drug costs. Platform contact information can also be used for patient-member feedback and satisfaction impact. Impact of the Platform on prescriber use of RTPB data in EHRs may also be measured as prescribers may likely learn to reduce patient call-backs by considering RTPB data in their EHRs prior to prescribing.

### 8. Platform Delegation

Patient-members may allow delegation of Platform communication to care-givers and other authorized parties.

### 9. Platform Specialty Engagement

Platform transactions for medications including specialty medications may trigger a message to pharmacists directly or through MedOps to engage members and providers including initiating Prior Authorizations, inserting Shared Patient Savings (SPS) where a portion of total drug costs savings are shared with the patient directly via VISA debit card or similar, etc.

### 10. Platform Secondary Prescriber Engagement

Platform messages can include highlighted information about high-cost medications with lower cost alternatives prescribed by non-Platform prescribers.

### 11. Platform Status Functionality

The Platform can be used to see current coverage and deductible status. The Platform can also be used to update patient-members on PA status or allow patient-members to identify a lower cost alternative which would waive the PA requirement and potentially trigger SPS. The Platform can be used to notify patient-members when a prescription is ready for pickup.

### 12. Platform as ‘Prescription Routing Service (RxRoute)

The Platform can duplicate the patient-member options that existed with paper prescriptions. The Platform can be configured so that a new prescription is held until the patient-member logs in to the MRG App and directs the Rx to a specific pharmacy. Alternatively, the prescription could be held pending patient direction for a pre-determined amount of time and then forwarded to the pharmacy selected by the prescriber if there is no patient-member activity. In certain examples, the duration of the on-hold period could be set by the patient-member, and an alternative pharmacy or site of service can be selected solely by the patient.

In certain examples, alternative medications may need prescriber approval. Alternative pharmacy or medication fulfillment may be assisted by pharmacists available via Platform services. In certain examples, formulary changes may occur and dictate which alternative medications are presented to the patient-member. In yet other examples, patient-members may choose to not have alternative medications and/or alternative pharmacies displayed to them.

### 13. Platform MedOps Integration

MedOps may allow users to see which patient-members had recent Platform engagement as well as the ability to search for members with recent Platform engagement. MedOps may also allow users to communicate with members using Platform member-selected communications methods (text, app, email, phone, mobile app). MedOps users can be notified in real-time when a Platform communication goes to one of their patient-members and a flag can be set to prioritize savings opportunities above a threshold. MedOps users engaging providers in follow-up to Platform communications can waive a Prior Authorization requirement for the provider who selects an alternative or meets PA criterion.

### 14. Platform Artificial Intelligence Analysis and MedOps

Platform transaction data may be compared to downstream claims and related outcomes data to identify optimal member messaging, data and follow up strategies and services. MedOps may be used by providers to identify patient-members who have recently received services and would best be assisted by health plan and/or provider follow up.

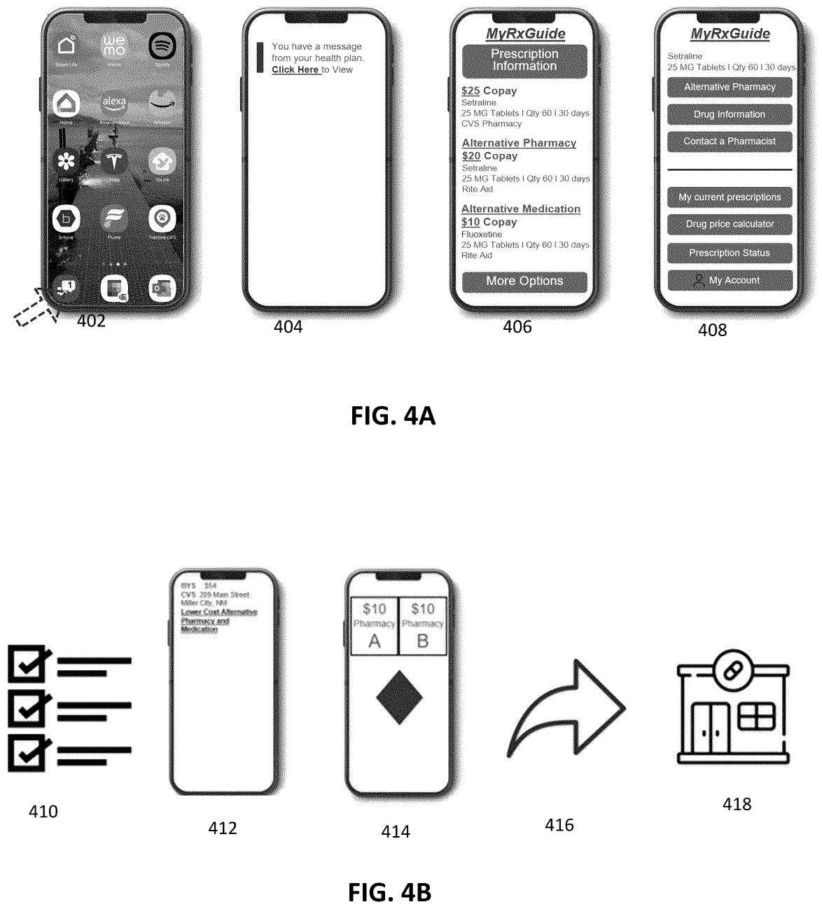

FIG. 4A depicts various views of a GUI of the drug-cost decision-support platform configured for patient-member usage.

As shown here, an icon for an app to access the GUI may be selected 402.

Next, the GUI may be displayed altering the user that there is a message to view 404.

Another view of the GUI may include Platform prescription information 406, which may include drug information from a default pharmacy, alternative pharmacy information, and alternative medication information (including drug name, dosage, quantities, supply duration, pharmacy name, and copay costs).

Another view of the GUI may include menu options for a given drug 408 including alternative pharmacy, drug information, contacting a pharmacist, current prescriptions for a patient-member, drug price calculator, prescription status, and patient-member account information.

FIG. 4B depicts prescription routing and other functionality of the drug-cost decision-support platform.

Illustrated here is a service within the Platform (“RxRoute”) for transferring unfilled prescriptions to a preferred pharmacy (which may be included in a prescription's information), requesting lower-cost alternatives, or placing prescriptions on hold, without an intervening prescriber.

As shown here, the workflow begins with 410, where a patient-member who is enrolled in MRG elects into RxRoute within the MRG GUI. Within this step, the patient-member must designate a default pharmacy for fulfillment (as highlighted in step 418).

Step 412 shows a text message at a display such as a smart phone of the patient-member, triggered by a new prescription, for patient-members enrolled in RxRoute. A link in the text message may direct to a secure landing page where patient-members can review alternative pharmacy options.

Step 414 represents the simple model of a mobile-optimized web site or web app for the Platform configured to allow a user such as the patient-member to swipe left or right on a preferred pharmacy. This election will re-route the prescription to the selected pharmacy 416. Additionally, if no pharmacy is selected within the configured allowable timeframe 418, the default pharmacy (selected in 410) will become the pharmacy of fulfillment.

Thus RxRoute advantageously allows the patient-member to:
1. Move an unfilled new prescription to a new pharmacy using an app or web service of the Platform without having to recontact the prescriber (change pharmacies).
2. Request prescriber consideration of a lower-cost alternative medication within the MRG app or web service of the Platform. Integration with EHRs may enable patient-generated messages to appear with the EHR workflow (patient messaging back to the prescriber).
3. Extend the period in which a prescription remains in RxRoute without being sent to a pharmacy. For example, the patient may be attempting to engage a prescriber to consider a lower-cost alternative medication or attempting to engage a pharmacist for consultation (patient hold on a prescription).

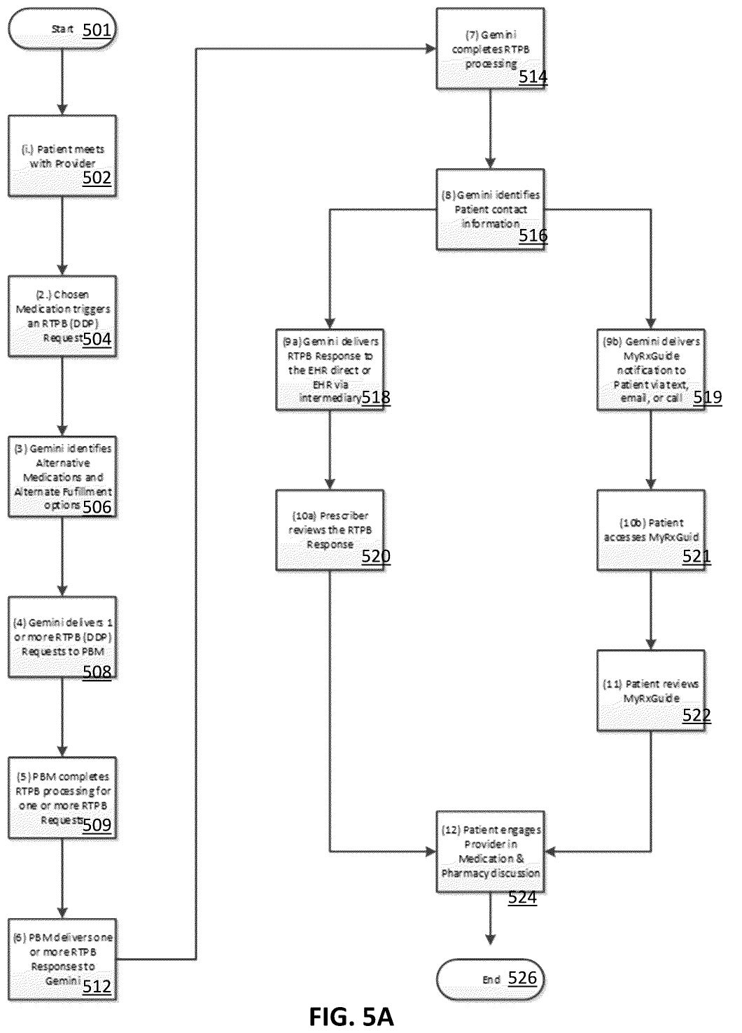

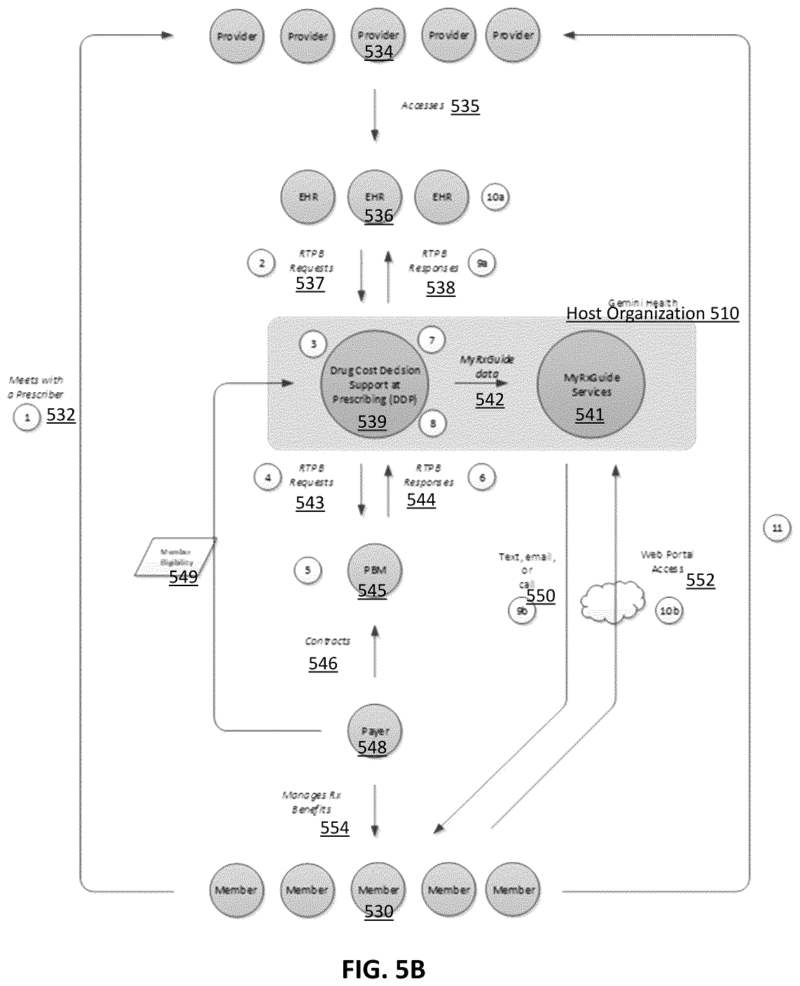

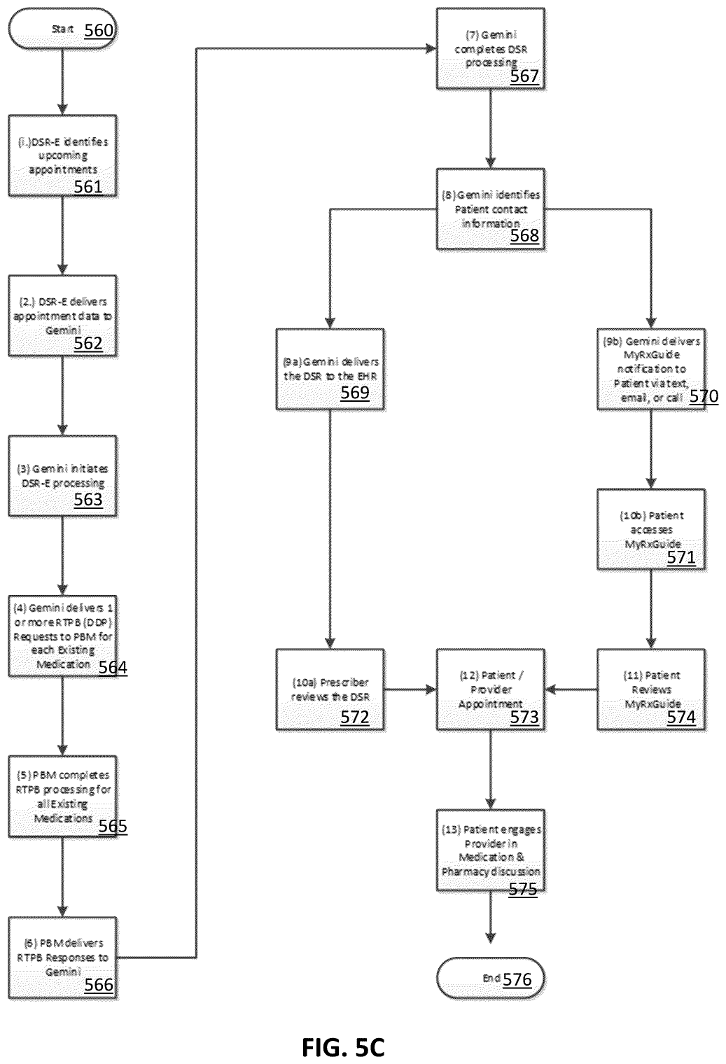

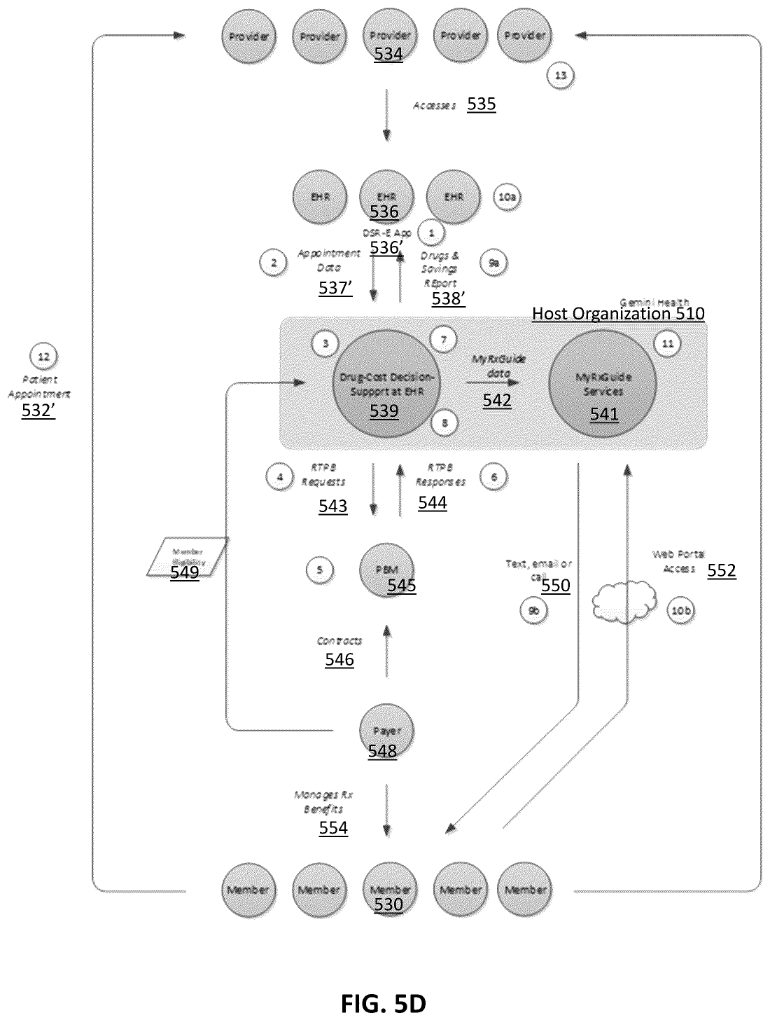

FIG. 5A depicts an exemplary workflow for the drug-cost decision-support platform at prescribing.

As shown here, the workflow begins 501 with a patient meeting with a provider 502 and a chosen medication triggering a Real-Time Prescription Benefit (RTPB) (DDP) request 405.

Next, the Platform identifies alternative medications and alternate fulfillment options 506.

The Platform may then deliver one or more RTPB (DDP) requests to a pharmacy benefit manager (PBM) 508. The PBM may be a third-party company that manages prescription drug benefits for health plans, employers and other clients acting as an intermediary between insurance providers and drug manufacturers.

The PBM may then complete RTPB processing for one or more RTPB requests 509.

Next, the PBM may deliver one or more RTPB responses to the Platform 512.

Next, the Platform may complete RTPB processing 514 and identify patient contact information 516.

In certain examples, the platform may in tandem or alternatively: a. deliver the RTPB response to the EHR directly or via an intermediary 518 and b. deliver a Platform notification to the patient via text, e-mail or telephone call 519.

Pursuant to the EHR receiving the delivered RTPB response from the Platform 518, a prescriber may review the RTPB response 520.

Pursuant to delivering the Platform notification to the patient 519, the patient may access the Platform (for example via a GUI) 521 and review the Platform 522.

Finally, the workflow may end 526 with the patient engaging their provider in a discussion regarding medication and pharmacy options 524.

FIG. 5B depicts another workflow of the drug-cost decision support platform at prescribing including user interactions.

As shown here, a member 530 may meet with a prescriber 532 such as a provider 534 who accesses 535 EHRs 536. EHRs 536 may then send RTPB requests 537 to Drug-Cost Decision Support at Prescribing (DDP) engine 539 at a host organization 510 or at the Platform. The DDP engine 539 may then send a RTPB to a PBM 545 that may send back RTPB responses 544 to the DDP engine 539. In certain examples, a payer 548 may send member eligibility data 549 to the DDP engine 539 and manage prescription benefits 554 for member(s) 530.

The DDP engine 539 may then transmit Platform data 542 to a Platform Services engine 541 which may also be at the host organization 510 or Platform.

Next, the Platform Services engine 541 may send a text, e-mail or telephone call 550 to one or more patient-members 530. Patient-member(s) 530 may then be given web portal access 552 to the Platform/host organization 510, for example via cloud services.

Finally patient-member(s) 530 may engage 531 providers 534 directly to discuss medication and pharmacy options.

FIG. 5C depicts a workflow of the drug-cost decision support platform at an electronic health record (EHR).

As shown here, the workflow beings 560 with Drug Savings Report at an Electronic health Record (DSR-E) identifying upcoming appointments for a patient/member 561. The DSR-E may then deliver appointment data to the Platform 562, at which point the Platform may initiate DSR-E processing 563.

The Platform may then deliver one or more RTPB (DDP) requests to a PBM for each existing medication for a patient 564. The PBM may then complete RTPB processing for all existing medications 565 followed by the PBM delivering RTPB responses to the Platform 566.

The Platform may then complete drug savings report (DSR) processing 567 and identify patient contact information 568. From there the Platform may deliver the DSR to the EHR 569 for a prescriber to review the DSR 572, resulting in a patient-member/provider appointment 573. Alternatively or in tandem, the Platform may deliver notification to the patient-member via text, e-mail, or phone call 570, pursuant to which the patient may access the Platform 571 and review 574, resulting in a patient/provider appointment 573 as previously described.

Following the patient-member/provider appointment 573, the workflow may end 576 with the patient-member engaging their provider in a medication and pharmacy discussion 575. In certain examples, the patient-member may bring a copy of the DSR to their appointment.

FIG. 5D depicts another workflow of the drug-cost decision support platform at an electronic health record (EHR) including user interactions.

As shown here, a patient-member 530 may have a patient appointment 532′ with a provider 534 who accesses 535 EHRs 536. A Drug-Cost Decision Support at EHR engine 539′ at a host organization 510 or at the Platform may send a drug savings report 538′ to the EHR 536, for example via a DSR-E app 595. EHRs 536 may then send appointment data requests 537′ to Drug-Cost Decision Support at EHR engine 539′ at a host organization 510 or at the Platform, for example via DSR-E app 595. The Drug-Cost Decision Support at EHR engine 539′ may then send a RTPB request 543 to a PBM 545 that may send back RTPB responses 544 to the Drug-Cost Decision Support at EHR engine 539′. In certain examples, a payer 548 may send patient-member eligibility data 549 to the Drug-Cost Decision Support at EHR engine 539′ and manage prescription benefits 554 for member(s) 530.

The Drug-Cost Decision Support at EHR engine 539′ may then transmit Platform data 542 to a Platform Services engine 541 which may also be at the host organization 510 or Platform.

Next, the Platform Services engine 541 may send a text, e-mail or telephone call 550 to one or more patient-members 530. Patient-member(s) 530 may then be given web portal access 552 to the Platform/host organization 510, for example via cloud services.

Finally, patient-member(s) 530 may engage 531 providers 534 directly to discuss medication and pharmacy options.

### Platform Workflows

In certain examples, patient-member appointments 532′ may trigger a variety of Platform messages, including:

### 1. Platform Trigger Messages

Platform trigger messages may be activated when a patient-member schedules a healthcare provider appointment. A provider's EHR, such as EHR 536, may use an Electronic Data Interchange (“EDI 270/271”) transaction to confirm patient-member's medical insurance eligibility 24 to 48 hours prior to an appointment.

### 2. Platform Matching

The Platform may intercept and interrogate the EDI 270 transaction to determine if the patient-member (patient-member number) and provider information (NPI) in the transaction matches the combination in the health plan's pharmacy claims file.

### 3. Platform Rules Engine

The Platform may use a customizable rules engine to determine if Platform communication and/or access is sent and/or if Platform data will be modified for the specific member. For example, if the NPI does not match the NPIs for the patient-member's prescriptions found in pharmacy claims, or there are no savings opportunities found in the DSR for the patient-member, the Platform data may be presented to the patient-member simply as a current medication list. The Rules engine may primarily focus on clinical and financial rules such as minimum savings required.

### 4. RTPB/Platform Data

The Platform may generate a Drugs and Savings Report (DSR) including the cost of the existing drugs in the claims file for the patient-member, and lower cost alternative drugs or fulfillment including (i.) 90 Days' Supply at Retail, (ii.) Mail Order, (iii.) Specialty Pharmacy, and/or 3rd parties such as CivicaScript, CostPlus/Cuban drugs etc. Information can include clinical information for patient-members as well.

Platform data can also include Cash Card options, payer-approved patient financial assistance, Shared Patient Savings and other patient-member benefits.

### 5. Additional Member Data

Optionally, the Platform-triggered DSR message such as DSR 538′, can include additional member data such as gaps in care (missing mammogram, overdue vaccination, etc.).

### 6. Platform Member Outreach

The Platform may send a text, email or mobile app notification to the patient-member enrolled in a Platform program with a hypertext link to the DSR 538′.

Alternatively, Platform member outreach can be delivered via telephone messages directing members to contact member services, get data via telephone or login to Platform/member portal or download the related Platform or payer Mobile App.

### 7. Platform Transaction Mapping

Platform transactions which result in DSR 538′ viewed by members may be recorded by the Platform or host organization for reporting purposes and matched to downstream claims to gauge impact.

### 8. Platform Reporting

The Platform may be configured to report monthly on the impact of the Platform as measured in changes to prescriptions which reduce drug costs. Platform contact information can also be used for member feedback and satisfaction impact. Impact of the Platform on prescriber use of RTPB data in EHRs may also be measured as prescribers may likely learn to reduce patient call-backs by considering RTPB data in their EHRs prior to prescribing.

### 9. Platform Delegation

Patient-members may allow delegation of Platform communication to care-givers and other authorized parties.

### 10. Platform Specialty Engagement

Platform transactions for medications including specialty medications may trigger a message to pharmacists directly or through MedOps to engage members and providers including initiating Prior Authorizations, inserting Shared Patient Savings (SPS), etc.

### 11. Platform Secondary Prescriber Engagement

Platform messages can include highlighted information about high-cost medications with lower cost alternatives prescribed by non-Platform prescribers.

### 12. Platform Status Functionality

The Platform can be used to see current coverage and deductible status. The Platform can also be used to update members on PA status or allow members to identify a lower cost alternative which would waive the PA requirement and potentially trigger SPS. The Platform can be used to notify members when a prescription is ready for pickup.

### 13. Platform MedOps Integration

MedOps may allow users to see which patient-members had recent Platform engagement as well as the ability to search for patient-members with recent Platform engagement. MedOps may also allow users to communicate with members using Platform member-selected communications methods (text, app, email, phone, mobile app). MedOps users can be notified in real-time when a Platform communication goes to one of their patient-members and a flag can be set to prioritize savings opportunities above a threshold. MedOps users engaging providers in follow-up to Platform communications can waive a Prior Authorization requirement for the provider who selects an alternative or meets PA criterion.

In yet other examples, new medication in a claim file may trigger a variety of Platform messages, including:

### 1. Platform Trigger Messages

Platform trigger messages may be activated when the patient-member has a new medication found in the health plan claims file.

### 2. Platform Matching

The Platform may intercept and interrogate the new medication to determine its Platform Score. The Platform score may rank medications based upon their historical likelihood of being switched to a lower cost alternatives and may further rank them by the savings/switch.

### 3. Platform Rules Engine

The Platform may use a customizable rules engine to determine if Platform communication and/or access is sent and/or if Platform data will be modified for the specific member. The Rules engine may primarily focus on clinical and financial rules such as minimum savings required and the new medication's Platform score. The Platform may also interrogate the information being sent to patients then trigger additional actions based upon Platform rules including AI. For example, a combination of factors such as medication type, savings available, geography, patient age, treatment diagnosis etc. may prompt an offer of pharmacist consultation and assistance.

### 4. RTPB/Platform Data

The Platform may generate a Drugs and Savings Report (DSR) including the cost of the existing drugs in the claims file for the patient-member, and lower cost alternative drugs or fulfillment including (i.) 90 Days' Supply at Retail, (ii.) Mail Order, (iii.) Specialty Pharmacy, and/or 3rd parties such as CivicaScript, CostPlus/Cuban drugs etc. Information can include clinical information for patient-members as well.

Platform data can also include Cash Card options, payer-approved patient-member financial assistance, Shared Patient Savings and other patient-member benefits.

### 5. Platform Member Outreach

The Platform may send a text or email to the enrolled patient-member with a hypertext link to the DSR 538′.

Alternatively, Platform member outreach can be delivered via telephone messages directing patient-members to contact member services, get data via telephone or login to Platform/member portal or download the related Platform or payer Mobile App.

### 6. Platform Transaction Mapping

Platform transactions which result in DSR 538′ viewed by patient-members may be recorded by the Platform or host organization for reporting purposes and matched to downstream claims to gauge impact.

### 7. Platform Reporting

The Platform may be configured to report monthly on the impact of the Platform as measured in changes to prescriptions which reduce drug costs. Platform contact information can also be used for member feedback and satisfaction impact. Impact of the Platform on prescriber use of RTPB data in EHRs may also be measured as prescribers may likely learn to reduce patient call-backs by considering RTPB data in their EHRs prior to prescribing.

### 8. Platform Delegation

Patient-members may allow delegation of Platform communication to care-givers and other authorized parties.

### 9. Platform Specialty Engagement

Platform transactions for medications including specialty medications may trigger a message to pharmacists directly or through MedOps to engage patient-members and providers including initiating Prior Authorizations, inserting Shared Patient Savings (SPS), etc.

### 10. Platform Secondary Prescriber Engagement

Platform messages can include highlighted information about high-cost medications with lower cost alternatives prescribed by non-Platform prescribers.

### 11. Platform Status Functionality

The Platform can be used to see current coverage and deductible status. The Platform can also be used to update patient-members on PA status or allow patient-members to identify a lower cost alternative which would waive the PA requirement and potentially trigger SPS. The Platform can be used to notify patient-members when a prescription is ready for pickup.

### 12. Platform MedOps Integration

MedOps may allow users to see which patient-members had recent Platform engagement as well as the ability to search for patient-members with recent Platform engagement. MedOps may also allow users to communicate with patient-members using Platform member-selected communications methods (text, app, email, phone, mobile app). MedOps users can be notified in real-time when a Platform communication goes to one of their patient-members and a flag can be set to prioritize savings opportunities above a threshold. MedOps users engaging providers in follow-up to Platform communications can waive a Prior Authorization requirement for the provider who selects an alternative or meets PA criterion.

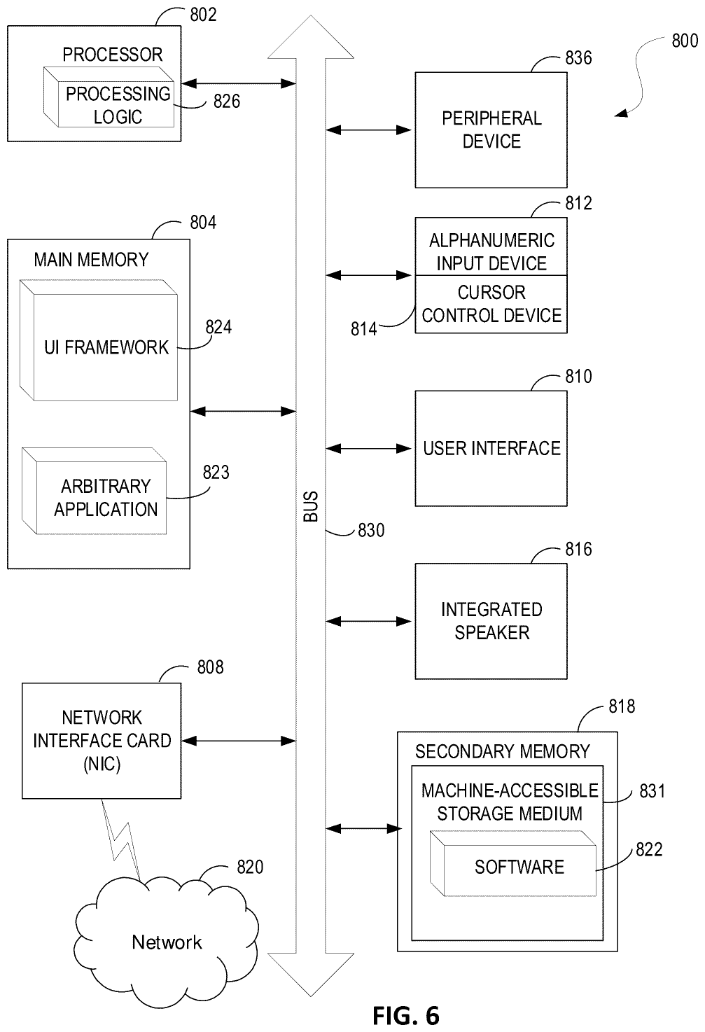

FIG. 6 illustrates a diagrammatic representation of a machine 600 in the exemplary form of a computer system, in accordance with one embodiment, within which a set of instructions, for causing the machine/computer system 600 to perform any one or more of the methodologies discussed herein, may be executed. In alternative embodiments, the machine may be connected (e.g., networked) to other machines in a Local Area Network (LAN), an intranet, an extranet, or the public Internet. The machine may operate in the capacity of a server or a client machine in a client-server network environment, as a peer machine in a peer-to-peer (or distributed) network environment, as a server or series of servers within an on-demand service environment. Certain embodiments of the machine may be in the form of a personal computer (PC), a tablet PC, a set-top box (STB), a Personal Digital Assistant (PDA), a cellular telephone, a web appliance, a server, a network router, switch or bridge, computing system, or any machine capable of executing a set of instructions (sequential or otherwise) that specify actions to be taken by that machine. Further, while only a single machine is illustrated, the term “machine” shall also be taken to include any collection of machines (e.g., computers) that individually or jointly execute a set (or multiple sets) of instructions to perform any one or more of the methodologies discussed herein.

The exemplary computer system 600 includes a processor 602, a main memory 604 (e.g., read-only memory (ROM), flash memory, dynamic random access memory (DRAM) such as synchronous DRAM (SDRAM) or Rambus DRAM (RDRAM), etc., static memory such as flash memory, static random access memory (SRAM), volatile but high-data rate RAM, etc.), and a secondary memory 618 (e.g., a persistent storage device including hard disk drives and a persistent database and/or a multi-tenant database implementation), which communicate with each other via a bus 630. Main memory 604 includes a UI Framework 624 to implement the mechanisms described herein, such as the foundational layer, providing components, features, the glue logic layer, and so forth. Arbitrary application 623 also of main memory 604 may be created or developed by the consumption of various features and their components as provided by the UI Framework 824. Main memory 604 and its sub-elements are operable in conjunction with processing logic 626 and processor 602 to perform the methodologies discussed herein. The computer system 600 may additionally or alternatively embody the server-side elements as described above.

Processor 602 represents one or more general-purpose processing devices such as a microprocessor, central processing unit, or the like. More particularly, the processor 602 may be a complex instruction set computing (CISC) microprocessor, reduced instruction set computing (RISC) microprocessor, very long instruction word (VLIW) microprocessor, processor implementing other instruction sets, or processors implementing a combination of instruction sets. Processor 602 may also be one or more special-purpose processing devices such as an application specific integrated circuit (ASIC), a field programmable gate array (FPGA), a digital signal processor (DSP), network processor, or the like. Processor 602 is configured to execute the processing logic 626 for performing the operations and functionality which is discussed herein.

The computer system 600 may further include a network interface card 608. The computer system 600 also may include a user interface 610 (such as a video display unit, a liquid crystal display (LCD), or a cathode ray tube (CRT)), an alphanumeric input device 612 (e.g., a keyboard), a cursor control device 614 (e.g., a mouse), and a signal generation device 616 (e.g., an integrated speaker). The computer system 600 may further include peripheral device 636 (e.g., wireless or wired communication devices, memory devices, storage devices, audio processing devices, video processing devices, etc.).

The secondary memory 618 may include a non-transitory machine-readable storage medium or a non-transitory computer readable storage medium or a non-transitory machine-accessible storage medium 631 on which is stored one or more sets of instructions (e.g., software 622) embodying any one or more of the methodologies or functions described herein. The software 622 may also reside, completely or at least partially, within the main memory 604 and/or within the processor 602 during execution thereof by the computer system 600, the main memory 604 and the processor 602 also constituting machine-readable storage media. The software 622 may further be transmitted or received over a network 620 via the network interface card 608.

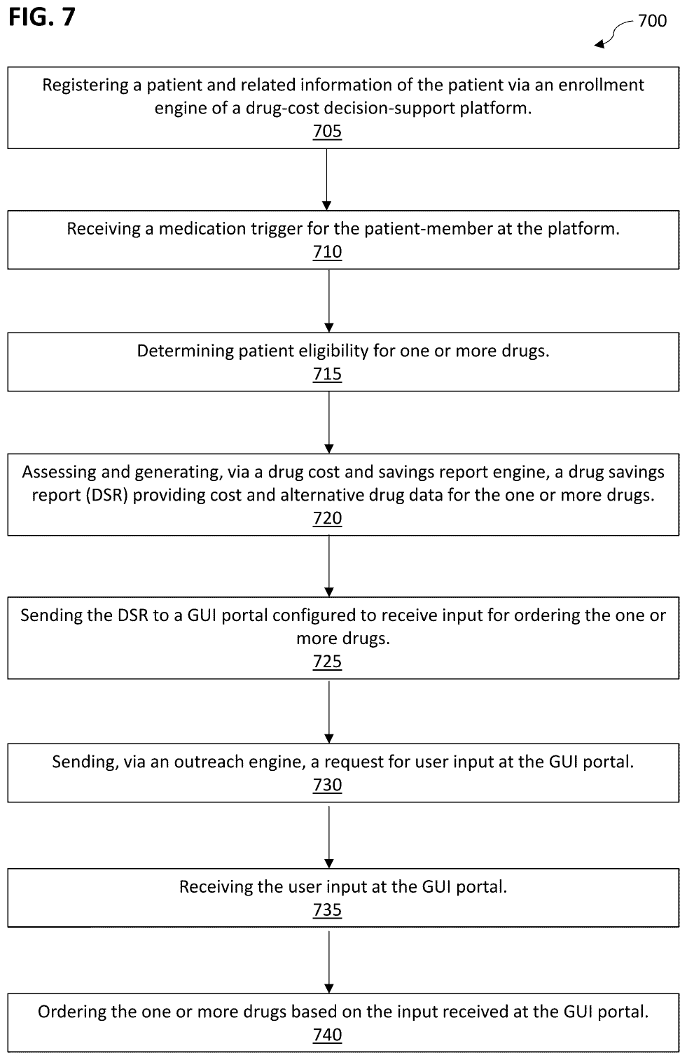

FIG. 7 is a flow chart illustrating a method for expedited and cost-effective prescription and ordering of medications using the drug-cost decision support platform 700.

Method 700 begins at step 705 with registering a patient and related information of the patient via an enrollment engine of a drug-cost decision support platform.

Next, method 700 includes receiving a medication trigger for the patient-member at the platform at step 710.

At step 715, patient eligibility for one or more drugs is determined.

At step 720, a drug cost and savings report engine assesses and generates a drug savings report (DSR) providing cost and alternative drug data for the one or more drugs.

At step 725, the DSR is sent to a GUI portal configured to receive input for ordering the one or more drugs.

At step 730, an outreach engine sends a request for user input at the GUI portal.

At step 735, user input is received at the GUI portal.

Method 700 concludes at step 740 with ordering the one or more drugs based on the input received at the GUI portal.

According to certain examples, method 700 further includes receiving the medication trigger based on one or more of: the related information of the patient, a prescription order, a real-time prescription benefit request, an EHR update, a request on behalf of the patient, a request on behalf of the healthcare provider, and a message from the platform to a pharmacist.

According to certain examples, method 700 further includes receiving the user input from one or more of: the patient, a healthcare provider, and a pharmacist.

According to certain examples, method 700 further includes accessing the GUI portal to provide the user input via one or more of: a member portal configured for the patient to access the DSR and an interface in the EHR configured for a healthcare provider to access the DSR; further in which accessing the GUI portal is achieved via a link sent via one or more of: a text message, a phone call and e-mail.

According to certain examples, method 700 further includes matching, via a matching engine, the related information of the patient including patient contact information and healthcare provider information with pharmacy claim information of a health plan for the patient.

According to certain examples, method 700 further includes incorporating clinical and financial rules in assessing and generating the DSR via a rules engine.

According to certain examples, method 700 further includes mapping DSR results to prescription claims via a transaction mapping engine to gauge impact of the platform.

According to certain examples, method 700 further includes integrating and notifying billing and prior authorization services via a billing services integration engine.

According to certain examples, method 700 further includes assessing and reporting status of one or more of: patient coverage, patient deductible, prior authorization, alternative drugs not requiring prior authorization and prescription ready for pick-up status via the drug cost and savings report engine.

### Use Cases

### I. Delivering a MRG Patient-Member Engagement Text

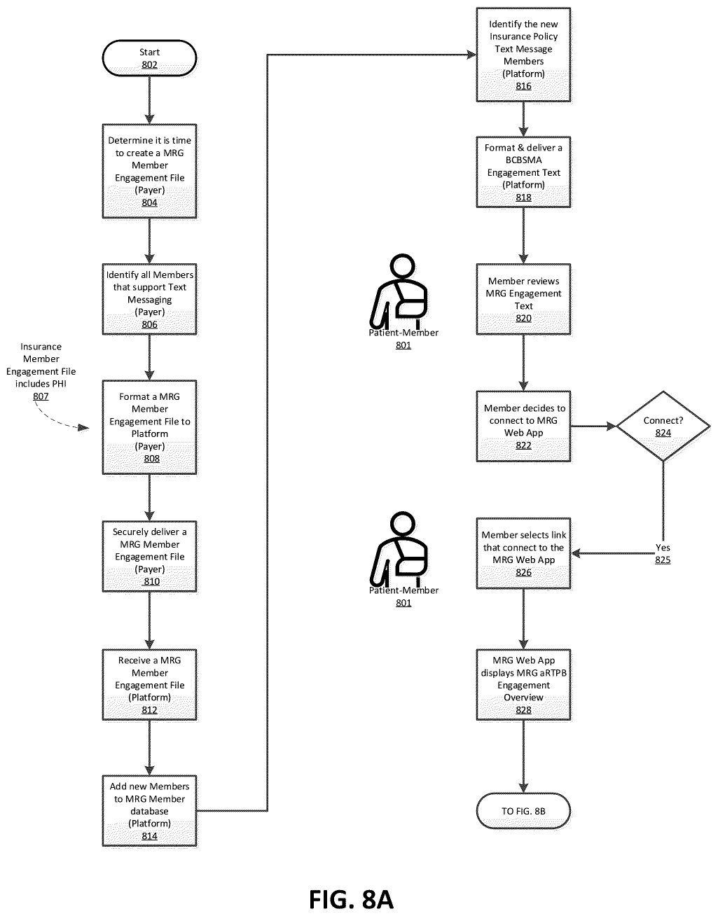

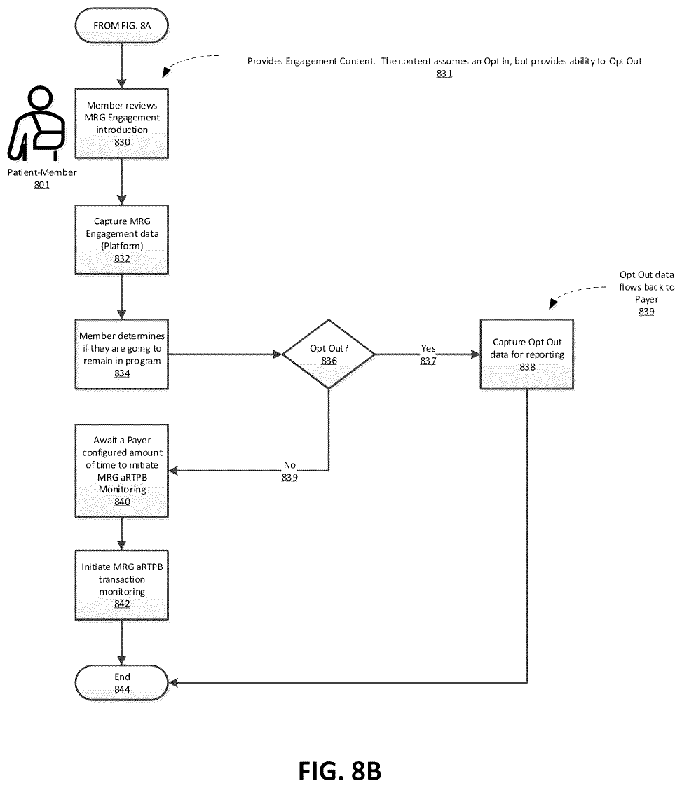

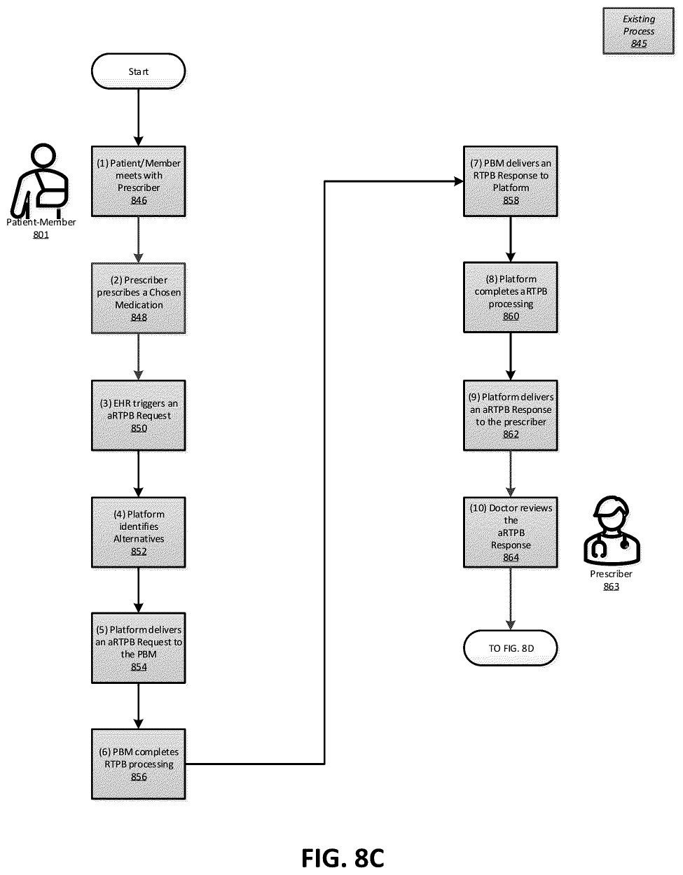

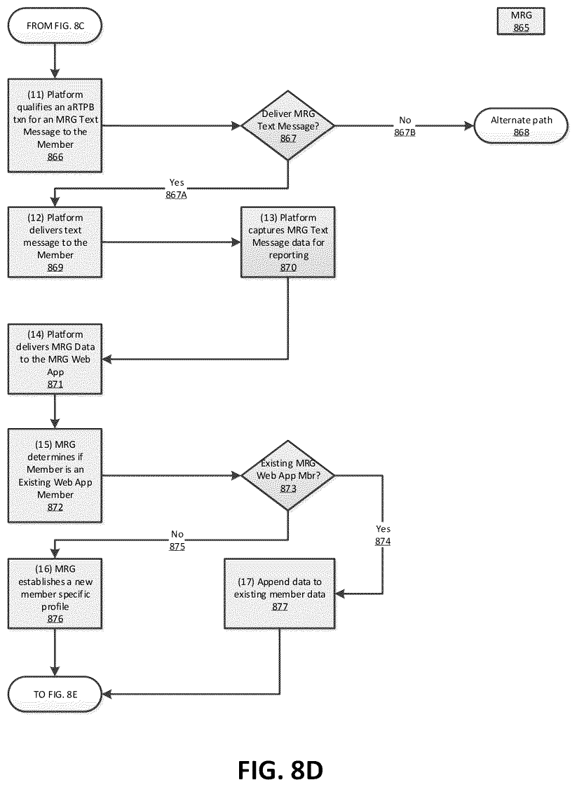

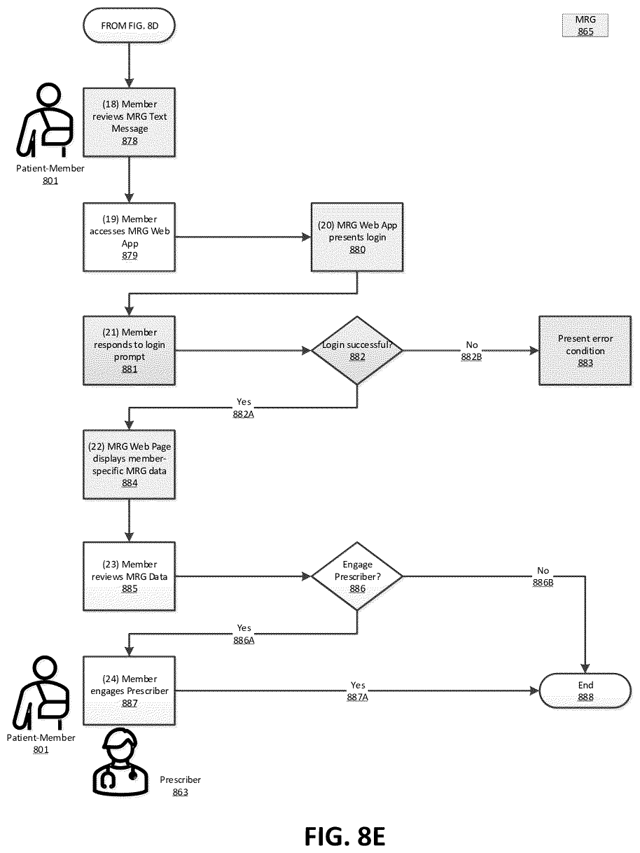

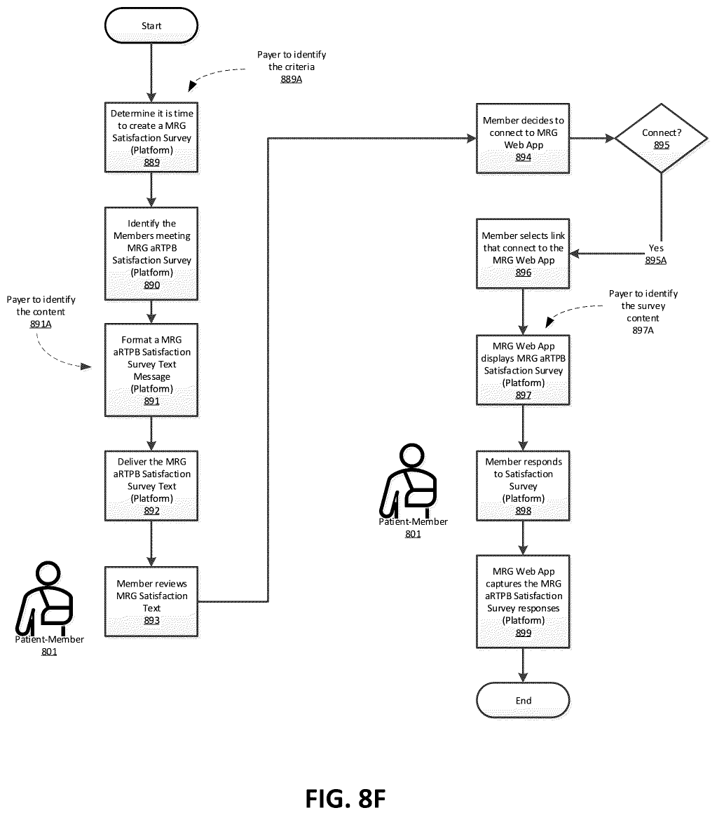

FIGS. 8A-8B illustrates a workflow for MRG Patient-Member Engagement.

Shown here is the process of a participating payer securely delivering an MRG Member Engagement file to the Platform and how the Platform receives the file, identifies new members, and delivers an MRG Member Engagement text message to the new patient-member, and how the new patient-member receives the text message, accesses the payer-specific Member Engagement webpage via a link in the text message, and reviews the web page content. Data is also captured for activity reporting.

As shown here, the process begins 802 with a payer determining that it is time to create a MRG Member Engagement File 804. The payer then identifies all members that support text messaging 806. Concurrently, an insurance policy member, such as Blue Cross Blue Shield of Massachusetts (BCBSMA), engagement file which may include protected health information (PHI) 807, may be formatted by the payer into a MRG Member Engagement File to the Platform 808. Next the payer may securely deliver a MRG Member Engagement File 810 to the Platform 812. The Platform may then add new members to the MRG Member database 814. The Platform may identify the new insurance policy text message members 816. The Platform may then format and deliver an insurance policy Engagement Text 818, with patient-member 801 reviewing the MRG Engagement Text 820. Patient-member 801 may the decide to connect to the MRG web app 722. If patient-member 801 decides yes 825 to connecting 824 to the web app, member 801 may select a link that connects to the MRG web app 826. Then, the MRG web app may display an MRG Real-Time Prescription Benefit Engagement (aRTPB) Overview 828.

The workflow continues at FIG. 8B.

As shown here, member 801 reviews MRG Engagement introduction 830, which provides Engagement Content 831, assuming an opt-in by member 801, but provides an ability for patient-member 801 to opt-out. The Platform may then capture MRG Engagement data 832, and patient-member 801 may determine if they are going to remain in the program 834. If patient-member 701 decides yes 837 to opting out 836, opt-out data may flow back to the Payer 839 and the opt-out data may be captured for reporting 838, ending the workflow 844. Alternatively, if patient-member 801 decides no 839 to opting-out 836, then there may be a Payer-configured amount of time to initiate MRG aRTPB monitoring 840. MRG aRTPB transaction monitoring may then be initiated 842, ending the workflow 844.

### II. Delivering an MRG Chosen Medication Text/Covered

In another use case, there is MRG-Engine processing for a patient-member that has consented to receive text messages, received an MRG Engagement text message, and did not opt-out as described in FIG. 8B, and has not received an MRG Chosen Medication Message that day (primary path) and a corresponding RTPB Transaction has chosen medication with a Coverage Status of ‘Covered.’ The Participating Payer may be configured to deliver one MRG Text Message per day per Payer/Member/Provider, and the RTPB Response for the chosen medication meets Qualifying MRG Transaction criteria, with or without a text message.

FIG. 8C illustrates MRG-aRTPB notifications under an existing process.

Process 845 begins with patient-member 801 meeting with a prescriber 846. Subsequent steps may include: the prescriber prescribing a chosen medication 848, EHR triggering an aRTPB request 850, the Platform identifying alternatives 852, the Platform delivering an aRTPB request to the PBM 854, and the PBM completing RTPB processing 856. Next, the PBM delivers an RTPB response to the Platform 858 and the Platform completes aRTPB processing 860. The Platform may then deliver an aRTPB Response to the prescriber 862. A doctor or prescriber 863 may then review the aRTPB Response 864.

FIGS. 8D-8E illustrates MRG-aRTPB notifications under the Platform/MRG 865.

As shown in FIG. 8D, the Platform may qualify an aRTPB transaction for an MRG text message to the patient-member at step 866. If an MRG text message 867 is delivered 867A, the Platform delivers text message to the patient-member at step 869. If the MRG text message is not delivered 867B, an alternate path 868 may be followed, and it may need to be determined if any data is captured. After the Platform delivers the text message to the patient-member at step 869, the Platform may capture the MRG text message data for reporting 870 and deliver MRG data to the MRG web app 871. The MRG may determine if the patient-member is an existing web app member 872/873. If yes 874, data may be appended to existing member data 877. In no 875, the MRG may establish a new member specific profile 876.

Continuing at FIG. 8E, the patient-member 801 may review the MRG text message 878, access MRG web app 879, with the MRG web app presenting a login 880. The patient-member may then respond to the login prompt 881. It may then be determined if the login is successful 882. If login is not successful 882B, an error condition may be presented 883. If login is successful 882A, the MRG web page may display member-specific MRG data 884. The patient-member 801 may then review the MRG data 885 and decide whether to engage a prescriber 886. If yes 886A, the member engages the prescriber 887/887A and the process ends 888. If the member does not decide to engage the prescriber 886B, the process also ends 888.

### III. Delivering an MRG Satisfaction Survey Text

In yet another use case example, a Participating Payer may securely deliver an MRG Member Engagement file to the Platform. The Platform may receive a file, identify new members, deliver an MRG Member Engagement text message to the new patient-member, and after receiving the text message the patient-member may access a Member Engagement webpage via a link in the text message, and review the web page content. Data may also be captured for activity reporting.

FIG. 8F illustrates a workflow for Platform/MRG Satisfaction.

The workflow starts with determining it is time to crease a MRG Satisfaction Survey (Platform) 889. The Payer may identify the criteria 889A. The Platform may then identify whether the members meet MRG aRTPB Satisfaction Survey criteria 890. The Platform may then format an MRG aRTPB Satisfaction Survey text message 891. The Payer may identify the content of the text message 891A. The MRG aRTPB Satisfaction Survey text message may be delivered to the patient-member at step 892, and patient-member 801 may review the MRG Satisfaction text 893. The patient-member 801 may decide to connect to an MRG web app 894. The patient-member 801 may decide whether to connect 895 to an MRG web app 894. If yes 895A, the patient-member 801 may select a link that connects to the MRG web app 896. The MRG web app may display an MRG aRTPB Satisfaction Survey 897, with the content of the survey being identified by the Payer 897A. The patient-member 801 may respond to the Satisfaction Survey 898. The workflow may end with the MRG web app capturing the MRG aRTPB Satisfaction Survey responses 899.

### IV. MyRxGuide @ Appointment (MRG-A)

According to certain examples, prescriber and prescriber representative may submit coverage eligibility transactions for upcoming office visits from practice management systems and/or EHRs. The Platform may access these transactions as they occur and determine covered eligibility for an identified service such as a healthcare professional/physician office visit, inpatient visit, or outpatient visit.

MRG-A processing may include validating that a patient is eligible for an identified medical service and other parameters such as whether the medical service is a qualifying medical service, that the medical service is for a future service date, that the patient hasn't received a DSR appointment within a certain time period, and that the patient agreed to opt into text messaging.

In certain examples, the MRG-A may then complete DSR processing for qualifying patients and existing medications, including identification of alternatives and patient costs for: existing medication, alternative medications, alternative fulfillment (extended days' supply and mail order), and pay-identified clinical messages. The DSR may then be delivered to the prescriber, for example via the EHR. DSR data may also be delivered to the Platform or web-app, with a text message formatted and delivered to the patient-member and capturing the data for real-time decision support and activity outcomes and reporting. The text message to the patient-member can be modified based upon the content of the DSR. For example, if the NPI does not match the NPIs for the patient-member's prescriptions found in pharmacy claims, or there are no savings opportunities found in the DSR for the patient-member, the Platform data may be presented to the patient-member simply as a current medication list.

The patient-member may then review the received text message and access the DSR on the Platform or web-app, and optionally print the DSR or bring a digital version of the DSR to their appointment.

In certain examples, MRG-A may interface and integrate with existing Platform and DSR solution capabilities, member engagement solutions and opt-out solutions, and may not require any changes to EHR or pharmacy benefit manager (PBM) data exchange communications. In yet other examples, the MRG-A may not support alternate fulfillment for alternative medications.

Described here is MRG-A processing for a patient that has agreed to receive payer supported healthcare text messages, has an existing medication profile, has scheduled an appointment, and has that has not received an MRG-A in a payer configured number of days. This MRG-A use case demonstrates the following:
1. The actors involved in the processing of an MRG-A
2. The dependencies between the actors and sequencing of events
3. The envisioned MRG-A processing rules and data that is captured
4. The Platform delivering an MRG Text Message to Patient
5. The patient receiving the MRG Text Message and accessing the Web App

According to certain examples, the Use Case describes a solution in which the Platform receives a copy of all Healthcare Eligibility Benefit Requests & Requests for a payer. An alternative approach may support the Platform receiving a subset of payer's Healthcare Eligibility Benefit Requests & Requests that are member or line-of-business specific or transactions specific to certain CPT codes.

### MRG-A Process Flow:

### 1. Patient:

a. Schedules an appointment

### 2. Prescriber Staff/Billing System:

a. Identifies the Patient
b. Ensures their healthcare benefit information is current
c. Identifies the contemplated service & date
d. Submits a Healthcare Benefit Inquiry (X12 270) to a Healthcare Clearinghouse

### 3. Healthcare Clearinghouse:

a. Receives a healthcare benefit inquiry (X12 270)
b. Validates the Patient eligibility
c. Validates the identified service and date of service
d. Formats a Healthcare Benefit Response (X12 271)
e. Delivers the Healthcare Benefit Response to the Prescriber/Billing System
f. Delivers a copy of the Healthcare Benefit Inquiry and Response to Platform

### 4. Platform:

a. Receives a Healthcare Benefit Inquiry and Response
b. Determines the payer identified to the transaction is active for MRG-A services
c. Determines that the Patient is pharmacy benefit eligible
d. Identifies the Patient's Existing Medications
e. Completes DSR processing (PBM RTPB processing)
f. Determine if a DSR was produced with a patient saving alternatives
i. Alternative Medications
ii. Alternate Fulfillment-Extended Days' Supply, and/or
iii. Alternate Fulfillment-Mail Order
g. Completes MRG-A processing
i. Identifies the DSR data
ii. Assigns a patient-specific link
iii. Identifies the payer's MRG Text Message format
iv. Identifies the Member/Patient's cellphone number
v. Formats an MRG-A Text Message
vi. Delivers the MRG-A Text Message

### 5. Patient:

a. Receives an MRG-A Text Message
b. Reviews the content
c. Access the Web App via the text message link

### 6. Platform:

a. Receives a request to access the Web App
b. Displays the MRG-A login

### 7. Patient-Member:

a. Reviews Web App login
b. Inputs the requested login data

### 8. Platform:

a. Validates MRG Web App login data
b. Presents the MRG-A DSR data
i. Existing Medications
ii. Alternative Medications with savings
iii. Alternate Fulfillment-Extended Days' Supply with savings
iv. Alternate Fulfillment-Mail with savings

### 9. Patient-Member:

a. Reviews MRG-A data
b. Captures, via cell phone or printing, the MRG-A data
c. Brings the MRG-A data to their appointment
d. Reviews the DSR alternates with the Prescriber

### Post-Conditions:

### 1. Patient:

a. Scheduled an appointment
b. Received an MRG-A Text Message
c. Reviewed the MRG-A Text Message
d. Accessed the Web App
e. Reviewed the DSR data with Prescriber

### 2. Prescriber Staff/Billing System:

a. Initiated a Healthcare Eligibility Benefit Request (X12 270, including benefit coverage eligibility)
b. Received a Healthcare Eligibility Benefit Response (X12 271)

### 3. Healthcare Benefit Clearinghouse:

a. Received a Healthcare Benefit Request (X12 270)
b. Adjudicated a Healthcare Benefit Response
c. Delivered a HealthCare Benefit Response (X12 271) to the Prescriber Staff/Billing Systems
d. Delivered a copy of the Healthcare Eligibility Request & Response to Platform

### 4. Platform:

a. Received a copy of the Healthcare Eligibility Benefit Request & Response
b. Determined the payer identified to the transaction is active for MRG-A services
c. Determined the Patient-Member is pharmacy benefit eligible
d. Identified the Patient-Member's Existing Medications
e. Completed DSR processing (PBM RTPB processing)
f. Determined if a DSR was produced with a patient saving alternatives
g. Completed MRG-A processing

In the following description, numerous specific details are set forth such as examples of specific systems, languages, components, etc., in order to provide a thorough understanding of the various embodiments. It will be apparent, however, to one skilled in the art that these specific details need not be employed to practice the embodiments disclosed herein. In other instances, well known materials or methods have not been described in detail in order to avoid unnecessarily obscuring the disclosed embodiments.

In addition to various hardware components depicted in the figures and described herein, embodiments further include various operations which are described below. The operations described in accordance with such embodiments may be performed by hardware components or may be embodied in machine-executable instructions, which may be used to cause a general-purpose or special-purpose processor programmed with the instructions to perform the operations. Alternatively, the operations may be performed by a combination of hardware and software.

Embodiments also relate to an apparatus for performing the operations disclosed herein. This apparatus may be specially constructed for the required purposes, or it may be a general purpose computer selectively activated or reconfigured by a computer program stored in the computer. Such a computer program may be stored in a computer readable storage medium, such as, but not limited to, any type of disk including floppy disks, optical disks, CD-ROMs, and magnetic-optical disks, read-only memories (ROMs), random access memories (RAMs), EPROMs, EEPROMs, magnetic or optical cards, or any type of media suitable for storing electronic instructions, each coupled to a computer system bus.

The algorithms and displays presented herein are not inherently related to any particular computer or other apparatus. Various general purpose systems may be used with programs in accordance with the teachings herein, or it may prove convenient to construct more specialized apparatus to perform the required method steps. The required structure for a variety of these systems will appear as set forth in the description below. In addition, embodiments are not described with reference to any particular programming language. It will be appreciated that a variety of programming languages may be used to implement the teachings of the embodiments as described herein.

Embodiments may be provided as a computer program product, or software, that may include a machine-readable medium having stored thereon instructions, which may be used to program a computer system (or other electronic devices) to perform a process according to the disclosed embodiments. A machine-readable medium includes any mechanism for storing or transmitting information in a form readable by a machine (e.g., a computer). For example, a machine-readable (e.g., computer-readable) medium includes a machine (e.g., a computer) readable storage medium (e.g., read only memory (“ROM”), random access memory (“RAM”), magnetic disk storage media, optical storage media, flash memory devices, etc.), a machine (e.g., computer) readable transmission medium (electrical, optical, acoustical), etc.

Any of the disclosed embodiments may be used alone or together with one another in any combination. Although various embodiments may have been partially motivated by deficiencies with conventional techniques and approaches, some of which are described or alluded to within the specification, the embodiments need not necessarily address or solve any of these deficiencies, but rather, may address only some of the deficiencies, address none of the deficiencies, or be directed toward different deficiencies and problems where are not directly discussed.

All publications and patent applications mentioned in this specification are herein incorporated by reference in their entirety to the same extent as if each individual publication or patent application was specifically and individually indicated to be incorporated by reference. Furthermore, it should be appreciated that all combinations of the foregoing concepts and additional concepts discussed in greater detail below (provided such concepts are not mutually inconsistent) are contemplated as being part of the inventive subject matter disclosed herein and may be used to achieve the benefits described herein.

Although illustrated as separate elements, the method steps described and/or illustrated herein may represent portions of a single application. In addition, in some embodiments one or more of these steps may represent or correspond to one or more software applications or programs that, when executed by a computing device, may cause the computing device to perform one or more tasks, such as the method step.

A person of ordinary skill in the art will recognize that any process or method disclosed herein can be modified in many ways. The process parameters and sequence of the steps described and/or illustrated herein are given by way of example only and can be varied as desired. For example, while the steps illustrated and/or described herein may be shown or discussed in a particular order, these steps do not necessarily need to be performed in the order illustrated or discussed.

The various exemplary methods described and/or illustrated herein may also omit one or more of the steps described or illustrated herein or comprise additional steps in addition to those disclosed. Further, a step of any method as disclosed herein can be combined with any one or more steps of any other method as disclosed herein.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under”, or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

In general, any of the apparatuses and methods described herein should be understood to be inclusive, but all or a sub-set of the components and/or steps may alternatively be exclusive and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

While the subject matter disclosed herein has been described by way of example and in terms of the specific embodiments, it is to be understood that the claimed embodiments are not limited to the explicitly enumerated embodiments disclosed. To the contrary, the disclosure is intended to cover various modifications and similar arrangements as are apparent to those skilled in the art. Therefore, the scope of the appended claims are to be accorded the broadest interpretation so as to encompass all such modifications and similar arrangements. It is to be understood that the above description is intended to be illustrative, and not restrictive. Many other embodiments will be apparent to those of skill in the art upon reading and understanding the above description. The scope of the disclosed subject matter is therefore to be determined in reference to the appended claims, along with the full scope of equivalents to which such claims are entitled.

What is claimed is:

1. A drug-cost decision-support platform, comprising:
an enrollment engine configured to enroll a patient and register patient-related information including one or more of: patient contact information, insurance and eligibility information, clinical information, hospital information, healthcare provider information, medical appointment information, hospital stay information, medication information including electronic health record (EHR) information, prescription information and health information for the patient;
a drug and savings report engine configured to generate one or more of: a drug savings report (DSR) and drug determination policies (DDP) providing cost and alternatives for one or more drugs prescribed for the patient;
an outreach engine configured to respond to a medication trigger with the generated one or more of the DSR and DDP; and
a graphical user interface (GUI) portal configured to display the one or more of the DSR and DDP and provide an ordering interface configured to order the one or more drugs.

2. The platform of claim 1, wherein the GUI portal configured to display the one or more of the DSR and DDP includes one or more of: a member portal and an interface in the EHR; further wherein the GUI portal is accessed via a link sent via one or more of: a text message, a phone call, e-mail and mobile app; further wherein one or more of: patient spoken language, patient printed language and engagement acknowledgement is used to display the one or more of the DSR and DDP.

3. The platform of claim 1, further comprising a matching engine configured to evaluate whether the patient-related information including the patient contact information and healthcare provider information matches pharmacy claim information of a health plan for the patient.

4. The platform of claim 1, further comprising a rules engine configured to incorporate clinical and financial rules in generating the one or more of the DSR and DDP.

5. The platform of claim 1, wherein the medication trigger is based on one or more of: the patient-related information, a prescription order, a real-time prescription benefit request, an EHR update, a request on behalf of the patient, a request on behalf of the healthcare provider, and a message from the platform to a pharmacist.

6. The platform of claim 1, wherein the one or more of the DSR and DDP further includes information from retailers, mail order, specialty pharmacies, and third-party pharmacies; wherein the one or more of the DSR and DDP further includes payment and financial assistance information.

7. The platform of claim 1, further comprising a reporting engine configured to report one or more of: patient satisfaction and impact of the platform in real-time prescribing behavior of healthcare providers based on reduced patient call-back for prescribing the one or more medications.

8. The platform of claim 1, further comprising a transaction mapping engine configured to match the one or more of the DSR and DDP results to prescription claims to gauge impact of the platform.

9. The platform of claim 1, further comprising a billing services integration engine configured to integrate with and send notification to billing and Prior Authorization services for medications billable to one or more of a pharmacy and medical benefit coverage eligibility.

10. The platform of claim 1, wherein the drug and savings report engine is further configured to assess and report status of one or more of: patient coverage, patient deductible, prior authorization, alternative drugs not requiring prior authorization and prescription ready for pick-up status.

11. The platform of claim 1, wherein prescription information includes a preferred pharmacy for the patient, further comprising a mechanism for one or more of: transferring unfilled prescriptions to the preferred pharmacy, requesting lower-cost drug alternatives, and placing prescriptions on hold.

12. A method for expedited and cost-effective digital prescription and ordering of medications, the method comprising:
registering a patient and related information of the patient via an enrollment engine of a drug-cost decision-support platform;
receiving a medication trigger for the patient at the platform;
determining patient eligibility for one or more drugs;
assessing and generating, via a drug cost and savings report engine, one or more of a drug savings report (DSR) and drug determination policies (DDP) providing cost and alternative drug data for the one or more drugs;
sending the one or more of the DSR and DDP to a graphical user interface (GUI) portal configured to receive input for ordering the one or more drugs;
sending, via an outreach engine, a request for user input at the GUI portal;
receiving the user input at the GUI portal; and
ordering the one or more drugs based on the input received at the GUI portal.

13. The method of claim 12, wherein the medication trigger is based on one or more of: the related information of the patient, a prescription order, a real-time prescription benefit request, an EHR update, a request on behalf of the patient, a request on behalf of the healthcare provider, and a message from the platform to a pharmacist.

14. The method of claim 12, further comprising receiving the user input from one or more of: the patient, a healthcare provider, and a pharmacist.

15. The method of claim 12, further comprising accessing the GUI portal to provide the user input via one or more of: a member portal configured for the patient to access the one or more of the DSR and DDP and an interface in the EHR configured for a healthcare provider to access the one or more of the DSR and DDP; further wherein accessing the GUI portal is achieved via a link sent via one or more of: a text message, a phone call and e-mail.

16. The method of claim 12, further comprising matching, via a matching engine, the related information of the patient including patient contact information and healthcare provider information with pharmacy claim information of a health plan for the patient.

17. The method of claim 12, further comprising incorporating clinical and financial rules in assessing and generating the one or more of the DSR and DDP via a rules engine.

18. The method of claim 12, further comprising matching the one or more of the DSR and DDP to prescription claims via a transaction mapping engine to gauge impact of the platform.

19. The method of claim 12, further comprising integrating and notifying billing and prior authorization services via a billing services integration engine.

20. The method of claim 12, further comprising assessing and reporting status of one or more of: patient coverage, patient deductible, prior authorization, alternative drugs not requiring prior authorization and prescription ready for pick-up status via the drug cost and savings report engine.

21. A method executing within a system of a host organization, the system having a processor and a memory therein to execute instructions within the system, wherein the method comprises:
registering a patient and related information of the patient via an enrollment engine of a drug-cost decision-support platform;
receiving a medication trigger for the patient at the platform;
determining patient eligibility for one or more drugs;
assessing and generating, via a drug cost and savings report engine, one or more of a drug savings report (DSR) and drug determination policies (DDP) providing cost and alternative drug data for the one or more drugs;
sending the one or more of the DSR and DDP to a graphical user interface (GUI) portal configured to receive input for ordering the one or more drugs;
sending, via an outreach engine, a request for user input at the GUI portal;
receiving the user input at the GUI portal; and
ordering the one or more drugs based on the input received at the GUI portal.

22. Non-transitory computer readable storage media having instructions stored thereon that, when executed by a processor of a system, the instructions cause the system to perform operations comprising:
registering a patient and related information of the patient via an enrollment engine of a drug-cost decision-support platform;
receiving a medication trigger for the patient at the platform;
determining patient eligibility for one or more drugs;
assessing and generating, via a drug cost and savings report engine, one or more of a drug savings report (DSR) and drug determination policies (DDP) providing cost and alternative drug data for the one or more drugs;
sending the one or more of the DSR and DDP to a graphical user interface (GUI) portal configured to receive input for ordering the one or more drugs;
sending, via an outreach engine, a request for user input at the GUI portal;
receiving the user input at the GUI portal; and
ordering the one or more drugs based on the input received at the GUI portal.
